(19) United States
(12) Patent Application Publication
(10) Pub. No.: US 2022/0209301 A1
(43) Pub. Date: Jun. 30, 2022

(54) CARBONATE SOLVENTS FOR NON-AQUEOUS ELECTROLYTES FOR METAL AND METAL-ION BATTERIES

(71) Applicant: SCE France, Lacq (FR)

(72) Inventors: MatjaZ KOZELJ, Billère (FR); Cécile PETIT, Billère (FR); Sabrina PAILLET, Lescar (FR); Karim ZAGHIB, Longueuil, Québec (CA)

(73) Assignee: SCE France, Lacq (FR)

(21) Appl. No.: 17/602,590

(22) PCT Filed: Apr. 15, 2020
(86) PCT No.: PCT/IB2020/053563

§ 371 Date: Oct. 8, 2021

(30) Foreign Application Priority Data
Apr. 16, 2019 (FR) 1904075

(51) Int. Cl. H01M 10/0569; H01M 10/0525; H01M 10/0568; H01M 10/0567

(52) U.S. Cl. CPC H01M 10/0569; H01M 10/0525; H01M 2300/0028; H01M 10/0567; H01M 10/0568

(57) ABSTRACT

There is provided a metal or metal-ion battery comprising an aluminum current collector and a low-corrosiveness non-aqueous electrolyte comprising, as a solvent, a carbonate compound of formula (I):

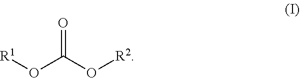

This battery has an upper voltage limit of about 4.2 V or more and anodic dissolution of aluminum during battery operation at said voltage is suppressed.

### FIELD OF THE INVENTION

The present invention relates to carbonate solvents for non-aqueous electrolytes for batteries. More specifically, the present invention is concerned with carbonate solvents for non-aqueous electrolytes that are characterized by their low corrosiveness against aluminum current collectors at voltages higher than 4.2 V vs. Li metal.

### BACKGROUND OF THE INVENTION

New technological solutions for telecommunications and especially electrification of transportation cells have been proposed for Li and Li-ion batteries. Their aim is to provide such batteries with the highest possible energy density in order to achieve higher voltage cathodes. This, however, requires high performance electrolytes which are resistant towards oxidation at the high potentials that occur during the operation of such a system. Also, other parasitic processes can cause deterioration and malfunctioning of the system. One such parasitic process is corrosion or electrolytic dissolution of the current collectors, which typically becomes significant at potentials beyond 4 V.

Conventional electrolytes used in most lithium and Li-ion battery systems, also in high voltage batteries, are based on LiPF₆ salt, which has many good properties. For example, it passivates the majority of aluminum current collector materials, has good conductivity and it is relatively chew. However, it also has some disadvantages, most notably sensitivity to moisture, causing HF to form, which causes rapid deterioration of battery performance. Another weakness is its limited thermal stability, limited solubility in polymers and emission of toxic decomposition products. The solvents used for the preparation of conventional electrolytes are cheaply available C₁-C₂ dialkyl carbonates and lower cyclic carbonates, most notably ethylene carbonate and propylene carbonate.

In order to substitute the risky LiPF₆ for safer alternatives, many salts have been proposed. One class of such salts are bissulfonyl amides; in fact, lithium bis(trifluoromethanesulfonyl)amide—LiTFSI, has been proposed as a salt for the preparation of electrolytes, including polymer electrolytes. In addition, other compounds of this class have been proposed. It has been discovered that LiTFSI produces serious anodic dissolution, erroneously called corrosion, of the aluminum current collector at voltages higher than 3.6 V. This means that the electrochemical charge that should be used for the charging of the battery is consumed for aluminum dissolution, such that the battery in fact cannot be charged. When this process occurs with a smaller rate (meaning only part of the charge is consumed by the corrosion process) the battery can be charged, but repeating the charging further dissolves the current collector. This slowly leads to diminished contact between the active electrode coating and the current collector, resulting in loss of capacity. This imposes a serious drawback for long-term operation, which entails many charges and discharges of the battery system.

For that reason, lithium bis(pentafluoroethanesulfonyl) amide—LiBETI was developed to overcome those problems, but its main disadvantages are its very high molecular weight, its high price and its accumulation in living organisms similar to all long chain perfluoroalkanes. On the other hand, two lighter salts have been proposed: lithium bis(fluorosulfonyl)amide—LiFSI and asymmetric lithium N-fluorosulfonyl-trifluoromethanesulfonyl amide—LiFTFSI. Other asymmetric bisfluorosulfonyl amides have also been suggested.

It has been stated that electrolytes containing LiFSI can support voltages up to 4.2V. However, it is not clear, and there is no experimental proof, that these electrolytes can support higher voltages.

Anodic dissolution of an aluminum current collector in sulfone-based solvents has been examined. As an alternative to molecular solvents, ionic liquids were reported as a good solvent for the suppression of anodic dissolution of aluminum. However, ionic liquids are not easily available, and their main drawback is their high price, which makes them less attractive for use in battery systems.

The influence of various solvents on anodic dissolution of aluminum collectors caused by LiTFSI has been examined. It has been found that collector corrosion depends on the electrolyte solvent, with strong corrosion in the presence of carbonates and lactones and minimal corrosion in the presence of nitriles. Such a conclusion discourages the utilisation of carbonate solvents for use with sulfonyl amide salts.

The inhibition of anodic dissolution of aluminum can be affected with fluoroborates, most effectively with lithium difluorooxalatoborate, LiDFOB; the drawback is the relatively high price of this additive.

Also, LiPF₆ can be used as an aluminum anodic dissolution inhibitor. However, very high concentrations of LiPF₆ are needed to effectively suppress the anodic dissolution of aluminum. In fact, due to the high concentrations, it would be more accurate to label such electrolytes as LiPF₆ electrolytes, with LiFSI as an additive to the LiPF₆.

Mother proposed solution is the use of highly concentrated electrolytes. However, there are several drawbacks, including the undesirable higher price of such a system and crystallisation problems at low temperatures.

There have also been more or less successful attempts to protect the aluminum current collector with a protective coating, but this increases the cost and weight of the battery. Furthermore, the most problematic point of this inhibition is that edges are not protected due to the cutting of electrodes to an appropriate size. Corrosion may propagate from the edges after many cycles, thus jeopardizing the long-term operation of such cells.

Possible solvents for lithium batteries have been discussed, but very little attention has been paid to unwanted proses on the current collectors. Most electrolytes used in the battery industry are based on the lower dialkyl carbonates: dimethyl, diethyl and ethylmethyl carbonate, mixed with additives, most notably ethylene carbonate. Syntheses of alkyl carbonates are very well developed, even on an industrial scale. Most suitable methods for their preparation in a laboratory are transesterifications.

Regarding high voltage applications, fluorinated carbonates have been proposed together with conventional LiPF₆ salt. However, these solvents are very expensive and can represent a serious environmental risk, like all long chin fluorinated compounds. Insecticidal activity of some fluorinated carbonates has also been described.

The above solutions to inhibit aluminum corrosion do not represent an optimal solution to the problem of anodic aluminum dissolution and therefore they do not represent an optimal replacement for conventional solvents.

### SUMMARY OF THE INVENTION

In accordance with the present invention, there is provided:
1. A metal or metal-ion battery comprising:
   (a) a cathode comprising an aluminum current collector and having an upper potential limit of about 4.2 V or more vs a Li-metal reference electrode,
   (b) an anode,
   (c) a separator membrane separating the anode and the cathode, and
   (d) a low-corrosiveness non-aqueous electrolyte in contact with the anode and the cathode,
   wherein the battery has an upper voltage limit of about 4.2 V or more,
   wherein anodic dissolution of aluminum in the aluminum current collector is suppressed during battery operation at voltages up to said upper voltage limit, and
   wherein the electrolyte comprises, as a solvent, a carbonate compound of formula (I):

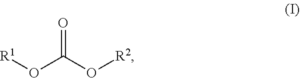

wherein:
R¹ represents a C₃-C₂₄ alkyl, a C₃-C₂₄ alkoxyalkyl, a C₃-C₂₄ ω-O-alkyl oligo(ethylene glycol), or a C₄-C₂₄ ω-O-alkyl oligo(propylene glycol), and
R² represents a C₁-C₂₄ alkyl, a C₁-C₂₄ haloalkyl, a C₂-C₂₄ alkoxyalkyl, a C₂-C₂₄ alkyloyloxyalkyl, a C₃-C₂₄ alkoxycarbonylalkyl, a C₁-C₂₄ cyanoalkyl, a C₁-C₂₄ thiocyanatoalkyl, a C₃-C₂₄ trialkylsilyl, a C₄-C₂₄ trialkylsilylalkyl, a C₄-C₂₄ trialkylsilyloxyalkyl, a C₃-C₂₄ ω-O-alkyl oligo(ethylene glycol), a C₄-C₂₄ ω-O-alkyl oligo(propylene glycol), a C₅-C₂₄ ω-O-trialkylsilyl oligo(ethylene glycol), or a C₆-C₂₄ ω-O-trialkylsilyl oligo(propylene glycol),
and a conducting salt dissolved in said solvent.
2. The battery of item 1, wherein the upper potential limit of the cathode is about 4.4 V or more, preferably about 4.6 V or more, about 4.8 V or more, about 5.0 V or more, about 5.2 V or more, about 5.4 V or more, or about 5.5 V or more, vs a Li-metal reference electrode.
3. The battery of item 1 or 2, wherein the upper voltage limit of the battery is about 4.4 V or more, preferably about 4.6 V or more, more preferably about 4.8 V or more, yet more preferably about 5.0 V or more, even more preferably about 5.2 V or more, more preferably about 5.4 V or more, or most preferably about 5.5 V or more.
4. The battery of any one of items 1 to 3, wherein R¹ represents a C₃-C₂₄ alkyl or a C₃-C₂₄ ω-O-alkyl oligo(ethylene glycol), preferably a C₃-C₂₄ alkyl.
5. The battery of any one of items 1 to 4, wherein R² represents a C₁-C₂₄ alkyl, a C₂-C₂₄ alkoxyalkyl, a C₁-C₂₄ cyanoalkyl, a C₄-C₂₄ trialkylsilyloxyalkyl, a C₅-C₂₄ ω-O-trialkylsilyl oligo(ethylene glycol), or a C₃-C₂₄ ω-O-alkyl oligo(ethylene glycol), preferably a C₁-C₂₄ alkyl.
6. The battery of any one of items 1 to 5, wherein the sum of the carbon atoms in R¹ and R² is:
   5 or more, preferably 6 or more, more preferably 7 or more, yet more preferably 8 or more, and most preferably 9 or more, and/or
   24 or less, preferably 20 or less, more preferably 16 or less, yet more preferably 14 or less, even more preferably 12 or less, and most preferably 10 or less.
7. The battery of any one of items 1 to 6, wherein R² is methyl or ethyl.
8. The battery of any one of items 1 to 7, wherein R¹ and/or R² is propyl, or isopropyl (2-propyl).
9. The battery of any one of items 1 to 8, wherein R¹ and/or R² is butyl, 2-butyl, 3-butyl, isobutyl (3-methylpropyl), or tertbutyl (2,2-dimethylethyl).
10. The battery of any one of items 1 to 9, wherein R¹ and/or R² is pentyl or one of its isomers (including 2-pentyl and 3-pentyl), 2-methylbutyl, 3-methylbutyl, 1-methyl-2-butyl, and 2-methyl-2-butyl).
11. The battery of any one of items 1 to 10, wherein R¹ and/or R² is hexyl or one of its isomers (including 2-hexyl and 3-hexyl), 2-methylpentyl, 3-methylpentyl, 4-methylpentyl, 3-methyl-2-pentyl, 4-methyl-2-pentyl, 2-methyl-2-pentyl, 2-methyl-3-pentyl, 3-methyl-3-pentyl, 3,3-dimethyl-2-butyl, 2,3-dimethyl-2-butyl, 2-ethylbutyl, and 3-ethyl-2-butyl).
12. The battery of any one of items 1 to 11, wherein R¹ and/or R² is heptyl, one of its isomers, or 2-ethylhexyl.
13. The battery of any one of items 1 to 12, wherein R¹ and/or R² is 2-methoxyethyl or 2-isopropoxyethyl.
14. The battery of any one of items 1 to 13, wherein R² is 2-cyanoethyl.
15. The battery of any one of items 1 to 14, wherein R² is (2-trimethylsilyloxy)ethyl.
16. The battery of any one of items 1 to 15, wherein R¹ and/or R² is 2-methoxyethyl, 2-isopropoxyethyl, or 2-(2-methoxyethoxy)ethyl.
17. The battery of any one of items 1 to 16, wherein R² is 2-trimethylsilyloxyethyl.
18. The battery of any one of items 1 to 17, wherein the carbonate compound of formula (I) is didodecyl carbonate, dibutyl carbonate, dipropyl carbonate, methyl propyl carbonate, diisopropyl carbonate, isopropyl methyl carbonate, ethyl dodecyl carbonate, ethyl propyl carbonate, ethyl isopropyl carbonate, diisobutyl carbonate, isobutyl methyl carbonate, dipentyl carbonate, methyl pentyl carbonate, di(2-ethylhexyl) carbonate, 2-ethylhexyl methyl carbonate, methyl 2-pentyl carbonate, di(2-pentyl) carbonate, 2-butyl methyl carbonate, di(2-butyl) carbonate, 2-ethylbutyl methyl carbonate, di(2-ethylbutyl) carbonate, isobutyl isopropyl carbonate, 2-cyanoethyl butyl carbonate, 2-methoxyethyl isobutyl carbonate, (2-trimethylsilyloxy)ethyl butyl carbonate, di(2-methoxyethyl) carbonate, 2-isopropoxyethyl methyl carbonate, di(2-isopropoxyethyl) carbonate, or di(2-(2-methoxyethoxy)ethyl) carbonate.
19. The battery of any one of items 1 to 18, wherein the compound of formula (I) is didodecyl carbonate, dibutyl carbonate, 2-ethylbutyl methyl carbonate, di(2-ethylbutyl) carbonate, di(2-butyl) carbonate, di(2-ethylhexyl) carbonate, 2-ethylhexyl methyl carbonate, di(2-pentyl) carbonate, ethyl dodecyl carbonate, 2-cyanoethyl butyl carbonate, 2-methoxyethyl isobutyl carbonate, (2-trimethylsilyloxy)ethyl butyl carbonate, di(2-isopropoxyethyl) carbonate, or diisobutyl carbonate.
20. The battery of any one of items 1 to 19, wherein the compound of formula (I) is didodecyl carbonate, di(2-ethylhexyl) carbonate, 2-ethylhexyl methyl carbonate, ethyl dodecyl carbonate, or diisobutyl carbonate, preferably diisobutyl carbonate.
21. The battery of any one of items 1 to 20, wherein the conducting salt is:
   LiClO₄;
   LiP(CN)_{α}F_{6-α}, where α is an integer from 0 to 6, preferably LiPF₆;
   LiB(CN)_{β}F_{4-β}, where β is an integer from 0 to 4, preferably LiBF₄;
   LiP(CₙF₂ₙ₊₁)_{γ}F_{6-γ}, where n is an integer from 1 to 20, and γ is an integer from 1 to 6;
   LiB(CₙF₂ₙ₊₁)_{δ}F_{4-δ}, where n is an integer from 1 to 20, and δ is an integer from 1 to 4;
   Li₂Si(CₙF₂ₙ₊₁)_{ε}F_{6-ε}, where n is an integer from 1 to 20, and ε is an integer from 0 to 6;
   lithium bisoxalato borate;
   lithium difluorooxalatoborate; or
   a compound represented by one of the following general formulas:

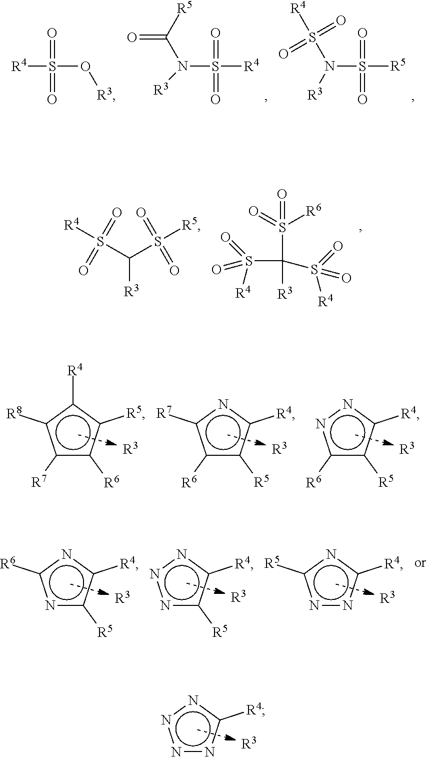

wherein:
R³ represents: Li, Na, K, Rb, Cs, Be, Mg, Ca, Sr, Ba, Al, hydrogen, or an organic cation; and
R⁴, R⁵, R⁶, R⁷, R⁸ represent cyano, fluorine, chlorine, branched or linear alkyl radical with 1-24 carbon atoms, perfluorinated linear alkyl radical with 1-24 carbon atoms, aryl, heteroaryl,
perfluorinated aryl, or heteroaryl;
or a derivative thereof.
22. The battery of any one of items 1 to 21, wherein the conducting salt is a sulfonylamide salt.
23. The battery of any one of items 1 to 22, wherein the conducting salt is a lithium salt, preferably a lithium sulfonylamide salt
24. The battery of item 23, wherein the lithium sulfonylamide salt is lithium bis(fluorosulfonyl)amide (LIFSI), lithium bis(trifluoromethanesulfonyl)amide (LiTFSI), or lithium N-fluorosulfonyl-trifluoromethanesulfonyl amide (LiFTFSI).
25. The battery of item 24, wherein the conducting salt is LiFSI.
26. The battery of any one of items 1 to 22, wherein the conducting salt is a sodium, a potassium, calcium, aluminum, or a magnesium salt
27. The battery of any one of items 1 to 26, wherein the conducting salt is present in the electrolyte at a concentration of at least about 0.05 M, at least about 0.1 M, at least about 0.5 M, or at least about 1 M, and/or at most about 3 M, at most about 2 M, at most about 1.5 M, or at most about 1 M.
28. The battery of item 27, wherein the concentration of the conducting salt in the electrolyte is 1 M.
29. The battery of any one of items 1 to 28, wherein the electrolyte further comprises one or more additives.
30. The battery of item 29, wherein the one or more additives are:
   an agent that improves solid electrolyte interphase and cycling properties;
   an unsaturated carbonate that improves stability at high and low voltages, and/or
   an organic solvent that diminishes viscosity and increases conductivity.
31. The battery of item 30, wherein the agent(s) that improve solid electrolyte interphase (SEI) and cycling properties and the unsaturated carbonate(s) together represents a total of at least about 0.1% w/w, at least 1% w/w, at least about 2% w/w, at least about 5% w/w, or at least about 7% w/w, and/or at most about 20% w/w, at most about 15% w/w, at most about 10% w/w, or at most about 7% w/w of the total weight of the electrolyte.
32. The battery of item 30 or 31, wherein the organic solvent(s) that diminishes viscosity and increases conductivity represents a total of at least about 1% v/v, at least about 2% v/v, at least about 5% v/v, or at least about 7% v/v, and/or at most about 80% v/v, at most about 50% v/v, at most about 20% v/v, at most about 15% v/v, at most about 10% v/v, or at most about 7% v/v of the total volume of the electrolyte.
33. The battery of any one of items 1 to 29, wherein the carbonate compound of formula (I) is the only solvent in the electrolyte.
34. The battery of any one of items 29 to 32, wherein the one or more additives are fluoroethylene carbonate (FEC), ethylene carbonate (EC), diethyl carbonate (DEC), or a mixture thereof.
35. The battery of item 34, wherein the one or more additives are FEC, preferably about 2 w/w % of FEC, alone or together EC, DEC or a mixture therefore, preferably alone or together with:
   about 5% v/v of EC,
   about 10% v/v of EC,
   about 15% v/v of EC,
   about 20% v/v of EC,
   about 30% v/v of EC,
   about 20% v/v of a mixture of EC and DEC,
   about 25% v/v of a mixture of EC and DEC,
   about 30% v/v of a mixture of EC and DEC,
   about 50% v/v of a mixture of EC and DEC,
   about 70% v/v of a mixture of EC and DEC, or
   about 75% v/v of a mixture of EC and DEC,
   wherein said mixture preferably has an EC:DEC volume ratio of from about 1:10 to about 1:1, preferably of about 3:7,
   all w/w % being based on the total weight of the electrolyte and all v/v % being based on the total volume of the electrolyte.
36. The battery of any one of items 1 to 35, wherein the carbonate compound of formula (I) the carbonate compound of formula (I) represents at least about 25% v/v, preferably at least about 50% v/v, more preferably at least about 75% v/v, yet more preferably at least about 85% v/v, even more preferably at least about 90% v/v, and most preferably at least about 95%, of the total volume of the electrolyte.
37. The battery of any one of items 1 to 36, wherein the electrolyte is free of corrosion inhibitors.
38. The battery of any one of items 1 to 36, wherein the electrolyte further comprises one or more corrosion inhibitors, such as LiPF6, lithium cyano fluorophosphates, lithium fluoro oxalatophosphates, LiDFOB, LiBF4, lithium fluoro cyanoborates, or LiBOB.
39. The battery of item 37, wherein the corrosion inhibitors represents a total of at least about 1% w/w, at least about 2% w/w, at least about 5% w/w, or at least about 10% w/w, and/or at most about 35% w/w, at most about 25% w/w, at most about 20% w/w, at most about 15% w/w, at most about 10% w/w, or at most about 7% w/w of the total weight of the electrolyte.
40. The battery of any one of items 1 to 38, being a lithium or a lithium-ion battery, preferably a lithium-ion battery.
41. The battery of item 40, wherein the anode is made of lithium metal or graphite.
42. The battery of items 40 or 41, wherein the cathode comprises a lithium compound disposed on the aluminum current collector, said lithium compound preferably being:
   a lithiated oxide of transition metal(s) such as LNO (LiNiO₂), LMO (LiMn₂O₄), LiCoₓNi₁₋ₓO₂ wherein x is from 0.1 to 0.9, LMC (LiMnCoO₂), LiCuₓMn₂₋ₓO₄, NMC (LiNiₓMn_{y}Co_{z}O₂), or NCA (LiNiₓCo_{y}Al_{z}O₂), or
   a lithium compound of transition metal(s) and a complex anion, such as LFP (LiFePO₄), LNP (LiNiPO₄), LMP (LiMnPO₄), LCP (LiCoPO₄), Li₂FCoPO₄; LiCo_{q}FeₓNi_{y}Mn_{z}PO₄, or Li₂MnSiO₄.
43. The battery of item 42, wherein the lithium compound is LMN or LCO.
44. The battery of any one of items 1 to 39, being a sodium battery, a sodium-ion battery, a potassium battery, a potassium-ion battery, a magnesium battery, a magnesium-ion battery, an aluminum battery, or an aluminum ion battery.
45. A carbonate compound of formula (I):

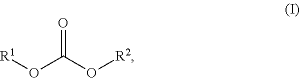

wherein
R¹ represents a C₃-C₂₄ alkyl, a C₃-C₂₄ alkoxyalkyl, a C₃-C₂₄ ω-O-alkyl oligo(ethylene glycol), or a C₄-C₂₄ ω-O-alkyl oligo(propylene glycol), and
R² represents a C₁-C₂₄ alkyl, a C₁-C₂₄ haloalkyl, a C₂-C₂₄ alkoxyalkyl, a C₂-C₂₄ alkyloyloxyalkyl, a C₃-C₂₄ alkoxycarbonylalkyl, a C₁-C₂₄ cyanoalkyl, a C₁-C₂₄ thiocyanatoalkyl, a C₃-C₂₄ trialkylsilyl, a C₄-C₂₄ trialkylsilylalkyl, a C₄-C₂₄ trialkylsilyloxyalkyl, a C₃-C₂₄ ω-O-alkyl oligo(ethylene glycol), a C₄-C₂₄ ω-O-alkyl oligo(propylene glycol), a C₅-C₂₄ ω-O-silyl oligo(ethylene glycol), or a C₆-C₂₄ ω-O-silyl oligo(propylene glycol),
with proviso that when R² is a C₁-C₉ alkyl, R¹ represents a C₁₀-C₂₄ alkyl, a C₃-C₂₄ alkoxyalkyl, a C₃-C₂₄ ω-O-alkyl oligo(ethylene glycol), or a C₄-C₂₄ ω-O-alkyl oligo(propylene glycol).
46. The carbonate compound of item 45, wherein, when R² is a C₁-C₉ alkyl, R¹ represents a C₃-C₂₄ alkoxyalkyl, a C₃-C₂₄ ω-O-alkyl oligo(ethylene glycol), or a C₄-C₂₄ ω-O-alkyl oligo(propylene glycol).
47. The carbonate compound of item 45 or 46, wherein the sum of the carbon atoms in R¹ and R² is:
   5 or more, preferably 6 or more, more preferably 7 or more, yet more preferably 8 or more, and most preferably 9 or more, and/or
   24 or less, preferably 20 or less, more preferably 16 or less, yet more preferably 14 or less, even more preferably 12 or less, and most preferably 10 or less.
48. The carbonate compound of any one of items 45 to 47, wherein R¹ represents a C₃-C₂₄ alkyl, a C₃-C₂₄ alkoxyalkyl, or a C₃-C₂₄ ω-O-alkyl oligo(ethylene glycol), preferably a C₃-C₂₄ alkyl or a C₃-C₂₄ alkoxyalkyl, and more preferably a C₃-C₂₄ alkyl.
49. The carbonate compound of any one of items 45 to 48, wherein R² represents a C₁-C₂₄ alkyl, a C₂-C₂₄ alkoxyalkyl, a C₁-C₂₄ cyanoalkyl, a C₄-C₂₄ trialkylsilyloxyalkyl, a C₅-C₂₄ ω-O-silyl oligo(ethylene glycol), or a C₃-C₂₄ ω-O-alkyl oligo(ethylene glycol), preferably a C₁-C₂₄ alkyl or a C₂-C₂₄ alkoxyalkyl, and more preferably a C₁-C₂₄ alkyl.
50. The carbonate compound of any one of items 45 to 49, wherein R¹ represents a C₁₀-C₂₄ alkyl (preferably C₁₂-C₂₄ alkyl, more preferably C₁₄-C₂₄ alkyl) and R² represents a C₁-C₂₄ alkyl.
51. The carbonate compound of any one of items 45 to 49, wherein R¹ represents a C₃-C₂₄ alkyl and R² represents a C₁-C₂₄ cyanoalkyl.
52. The carbonate compound of item 49, wherein the cyanoalkyl is 2-cyanoethyl.
53. The carbonate compound of any one of items 45 to 49, wherein R¹ represents a C₃-C₂₄ alkyl and R² represents a C₂-C₂₄ alkoxyalkyl.
54. The carbonate compound of any one of items 45 to 49, wherein R¹ represents a C₃-C₂₄ alkoxyalkyl and R² represents a C₁-C₂₄ alkyl.
55. The carbonate compound of any one of items 45 to 49, wherein R¹ and R² both represent a C₃-C₂₄ alkoxyalkyl.
56. The carbonate compound of any one of items 53 to 55, wherein the alkoxyalkyl is 2-methoxyethyl or 2-isopropoxyethyl.
57. The carbonate compound of any one of items 45 to 49, wherein R¹ represents a C₃-C₂₄ alkyl and R² represents a C₄-C₂₄ trialkylsilyloxyalkyl.
58. The carbonate compound of item 57, wherein the trialkylsilyloxyalkyl is (2-trimethylsilyloxy)ethyl.
59. The carbonate compound of any one of items 45 to 49, wherein R¹ and R² both represent a C₃-C₂₄ ω-O-alkyl oligo(ethylene glycol).
60. The carbonate compound of item 59, wherein the ω-O-alkyl oligo(ethylene glycol) is 2-(2-methoxyethoxy)ethyl.
61. The carbonate compound of any one of items 45 to 49, wherein the carbonate compound of formula (I) is didodecyl carbonate, ethyl dodecyl carbonate, 2-cyanoethyl butyl carbonate, 2-methoxyethyl isobutyl carbonate, (2-trimethylsilyloxy)ethyl butyl carbonate, di(2-methoxyethyl) carbonate, 2-isopropoxyethyl methyl carbonate, di(2-isopropoxyethyl) carbonate, or di(2-(2-methoxyethoxy)ethyl) carbonate.
62. The carbonate compound of item 61, wherein the compound of formula (I) is didodecyl carbonate, ethyl dodecyl carbonate, 2-cyanoethyl butyl carbonate, 2-methoxyethyl isobutyl carbonate, (2-trimethylsilyloxy)ethyl butyl carbonate, or di(2-isopropoxyethyl) carbonate.
63. The carbonate compound of item 62, wherein the compound of formula (I) is didodecyl carbonate, or ethyl dodecyl carbonate.

### BRIEF DESCRIPTION OF THE DRAWINGS

In the appended drawings:

FIG. 1 shows the chronoamperometry of an aluminum current collector versus Li metal at potentials increasing from 4 to 5.5 V by 0.1 V steps, 1 h at each step, in a conventional electrolyte comprising LIFSI and EC/DEC;

FIG. 2 shows the chronoamperometry of an aluminum current collector versus Li metal at potentials increasing from 4 to 5.5 V by 0.1 V steps, 1 h at each step, in a conventional electrolyte comprising LIFTFSI and EC/DEC;

FIG. 3 shows the chronoamperometry of an aluminum current collector versus Li metal at potentials increasing from 4 to 5.5 V by 0.1 V steps, 1 h at each step, in a conventional electrolyte comprising LITFSI and EC/DEC;

FIG. 4 shows the chronoamperometry of an aluminum current collector versus Li metal at potentials increasing from 4 to 5.5 V by 0.1 V steps, 1 h at each step, in an electrolyte according to an embodiment of the present invention comprising LIFSI and diisobutyl carbonate;

FIG. 5 shows the chronoamperometry of an aluminum current collector versus Li metal at potentials increasing from 4 to 5.5 V by 0.1 V steps, 1 h at each step, in an electrolyte according to an embodiment of the present invention comprising LIFTFSI and diisobutyl carbonate;

FIG. 6 shows the chronoamperometry of an aluminum current collector versus Li metal at potentials increasing from 4 to 5.5 V by 0.1 V steps, 1 h at each step, in an electrolyte according to an embodiment of the present invention comprising LITFSI and diisobutyl carbonate;

FIG. 7 shows the charge/discharge curves of an LCO cathode versus Li metal in a conventional electrolyte comprising LIFSI and EC/DEC;

FIG. 8 shows the charge/discharge curves of an LCO cathode versus Li metal in an electrolyte according to an embodiment of the present invention comprising LIFSI and diisobutyl carbonate;

FIG. 9 shows the charge/discharge curves of an LCO cathode versus Li metal in an electrolyte according to an embodiment of the present invention comprising LIFSI and diisobutyl carbonate+EC;

FIG. 10 shows the charge/discharge curves of an LMN cathode versus Li metal in a conventional electrolyte comprising LIFSI and EC/DEC;

FIG. 11 shows the charge/discharge curves of an LMN cathode versus Li metal in an electrolyte according to an embodiment of the present invention comprising LIFSI and diisobutyl carbonate;

FIG. 12 shows the discharge capacity of three cells versus cycle number, the first cell using LiFSI in diisobutyl carbonate, the second cell using LiFSI in 90% diisobutyl carbonate/10% EC, and the third cell using a conventional electrolyte of 1 M LiPF6 in EC/DEC (3:7 vol).

### DETAILED DESCRIPTION OF THE INVENTION

The present inventors have found that carbonate compounds of formula (I) can advantageously be used as solvents in non-aqueous electrolytes in batteries comprising a one cathode comprising an aluminum current collector because they are characterized by their low corrosiveness against aluminum, even at voltages of or higher than 4.2 V, even in electrolytes containing lithium sulfonylamide salts. Utilization of these lithium sulfonylamide salts with conventional solvents in such high voltage systems is typically not possible as anodic dissolution of aluminum becomes the preferred electrochemical reaction and the vast majority of the charge is consumed for this detrimental corrosion process.

While the low corrosiveness of the carbonate compounds of formula (I) is especially advantageous when lithium sulfonylamide salts are the main conducting salt, the person skilled in the art would recognize the potential of these solvents for achieving high voltage lithium and lithium ion batteries in connection with other conducting salts. The skilled person would also understand that the electrolyte can be used in different types of batteries, such as sodium, potassium, calcium, aluminum and magnesium-based batteries, and that when doing so, other salts can be dissolved in the solvents, for example sodium, potassium, calcium, aluminum and magnesium salts.

Indeed, the carbonate compounds of formula (I) are characterized by their capacity to suppress anodic dissolution of aluminum (e.g. in an aluminum current collector as well as any other aluminum member in the battery) when used as solvents in electrolytes in batteries, even at potentials higher than 4.2 V, as measured vs a lithium metal reference electrode. Such examples of batteries are a lithium or lithium-ion batteries. For clarity, unless specified otherwise, all electrode potentials in the present application are referenced to a Li metal anode.

Anodic dissolution of the aluminum current collector is defined as the dissolution of an aluminum current collector at a certain externally forced potential (the critical potential), which is higher than the open circuit potential. At the critical potential, the components of the electrolyte react with the surface of the collector and form soluble compounds, which in turn dissolve in the electrolyte and cause dissolution of the aluminum i.e. quasi corrosion. Significant dissolution of the aluminum can lead to malfunctioning of the battery system, if its operating voltage surpasses the critical potential. Accordingly, suppressing anodic dissolution enables safer and more powerful battery technologies, especially lithium-ion batteries. Herein, “suppressing” anodic dissolution means that anodic dissolution does not occur or that is reduced to such a level that it becomes non-deleterious to the battery.

This low corrosiveness of the carbonate compounds of formula (I) also enables the manufacture of batteries containing aluminum current collectors with extended operating voltages (in particular, operating voltages over 4.2 V vs a Li-metal reference electrode), even for electrolytes containing lithium sulfonylamide salts and lithium-ion batteries. This allows for the preparation of non-aqueous electrolytes containing lithium sulfonylamide salts that are free of corrosion inhibitors (while still maintaining said low corrosiveness against aluminum current collectors at voltages higher than 4.2 V).

Furthermore, when compared to conventional carbonate solvents (e.g. ethylene carbonate (EC), diethyl carbonate (DEC), and the like), the carbonate compounds of formula (I) have a wider operating temperature range, especially when used in lithium-ion batteries. Indeed, the temperature range in which the carbonate compounds of formula (I) are liquid (without crystallization) tends to be wider than that of these conventional carbonate solvents. For example, in embodiments, the carbonate compounds of formula (I) can have a melting point well below −10° C., and, in some cases, do not even have a melting point and thus stay liquid, without crystallizing, until they reach their glass transition point. Further, the melting point of the carbonate compounds tends to decrease with growing molecular mass to a certain point.

Further, the carbonate compounds of formula (I) have a higher boiling point than conventional carbonate solvents. For example, the boiling points of dimethyl carbonate, diethyl carbonate, dipropyl carbonate and dibutyl carbonate are 90, 126, 168 and 207° C., respectively. This indicates that electrolytes prepared from higher carbonates can be used at higher temperatures without the risk of rapid evaporation. These higher boiling points translates into improved safety properties for the batteries containing the electrolyte using the carbonate compounds of formula (I) as solvent.

Finally, the carbonate compounds of formula (I) are a very green, that is environmentally benign, group of solvents.

The inventors thus provide herein a metal or metal-ion battery comprising:
(a) a cathode comprising an aluminum current collector and having an upper potential limit of about 4.2 V or more vs a Li-metal reference electrode,
(b) an anode,
(c) a separator membrane separating the anode and the cathode, and
(d) a low-corrosiveness non-aqueous electrolyte in canted with the anode and the cathode,
   wherein the battery has an upper voltage limit of about 4.2 V or more,
   wherein anodic dissolution of aluminum in the aluminum current collector is suppressed during battery operation at voltages up to said upper voltage limit, and
   wherein the electrolyte comprises, as a solvent, a carbonate compound of formula (I) and a conducting salt dissolved in said solvent.

In embodiments, the upper potential limit of the cathode is preferably about 4.4 V or more, about 4.6 V or more, about 4.8 V or more, about 5.0 V or more, about 5.2 V or more, about 5.4 V or more, or about 5.5 V or more, vs a Li-metal reference electrode.

In embodiments, the upper potential limit of the cathode is preferably about 6.0 V or less, about 5.6 V or less, about 5.5 V or less, about 5.4 V or less, about 5.2 V or less, about 5.0 V or less, about 4.8 V or less, vs a Li-metal reference electrode.

In embodiments, the upper voltage limit of the battery is preferably about 4.4 V or more, about 4.6 V or more, about 4.8 V or more, about 5.0 V or more, about 5.2 V or more, about 5.4 V or more, or about 5.5 V or more.

In embodiments, the upper voltage limit of the battery is preferably about 6.0 V or less, about 5.6 V or less, about 5.5 V or less, about 5.4 V or less, about 5.2 V or less, about 5.0 V or less, about 4.8 V or less.

The lower potential limit of the cathode and the lower voltage limit of the battery are not substantially affected by using a carbonate compound of formula (I) as a solvent in the battery of the invention. Indeed, these lower limits are not critical to the invention since anodic dissolution occurs only at elevated potentials. Furthermore, the lower potential limit of cathode it typically not affected by the solvent used for the electrolyte. Therefore, these lower limits will be those found in corresponding conventional batteries that use other solvents.

Indeed, cathodes are characterized by a potential window that goes from a lower potential limit to an upper potential limit. The lower potential limit is the potential beyond which further discharge would harm the cathode. The upper potential limit is the potential beyond which further charge would harm the cathode. These potential values are always given referring to a certain reference. For example, for all lithium batteries, this reference is a Li-metal reference electrode. Herein, the potential values all refer to a Li-metal reference electrode, even when referring to other types of batteries (sodium-based, magnesium-based batteries, etc.).

Furthermore, batteries are characterized by an operating voltage window that goes from a lower voltage limit to an upper voltage limit. The lower voltage limit is the voltage at which a battery is considered fully discharged and beyond which further discharge would harm the battery (or its components). The upper potential limit is the voltage at which a battery is considered fully charged and beyond which further charge would harm the battery (or its components). Therefore, batteries are operated at voltages within their operating voltage window, i.e. they are charged/discharged so that their voltage falls within their operating voltage window, ideally as close as possible to the upper voltage limit when they are charged so as to provide a maximum of energy.

For note, a battery voltage is the difference between the potential of the cathode and that of the anode.

Battery voltage=(potential of the cathode)−(potential of the anode)

As such, no reference is needed when providing a battery voltage value.

When the anode of the battery is lithium metal, it has (all the time) a potential of 0V. Thus, in such cases, the upper and lower voltage limits of the battery are equal to the upper and lower potential limits of the cathode. In other cases, such as when the anode is made of graphite, the anode has a potential >0V. When the anode has a potential >0V, the upper and lower voltage limits of the battery are lower than the upper and lower potential limits of the cathode, respectively. For example, a graphite anode has (most of the time) a potential of about 0.1V, but this potential can nevertheless vary from about 2.5V to very close to 0V. An LTO anode has a potential of about 1.5V most of the time.

As noted above, anodic dissolution of aluminum in the aluminum current collector is suppressed during battery operation at voltages at least up to said upper voltage limit. Herein, the “suppression” of anodic dissolution mews that this phenomenon does not take place at all or that it is so limited that the battery can be charged up to said upper voltage without losing significant part of charge for anodic dissolution of aluminium. For example, less than 0.01%, preferably less than 0.001%, and more preferably less than 0.0001% of the charge is lost. In another scale, it is preferable that the corrosion current density be lower than about 1 microA/cm². Indeed, when significant anodic dissolution occurs within the operating voltage window of a battery, significant amount of charge is lost, and significant amount of aluminium is dissolved and contact between the current collector and active material lost, further leading into loss of useful capacity. In the most catastrophic scenario, most of the charge is consumed by aluminium dissolution when first charging the battery, which means that the amount of charge stored by the battery (i.e. the amount of useful charge) is very small. In other words, the battery does not work. In particular, electrolytes comprising bis(sulfonylamide) salts (of e.g. lithium or of other metals in batteries based on other metals) can cause such catastrophic anodic dissolution of aluminium. In contrast, when such salts are dissolved in a carbonate compound of formula (I), as a solvent, the anodic dissolution is successfully suppressed.

In preferred embodiments, the battery is a lithium battery, a lithium-ion battery, sodium battery, a sodium-ion battery, a potassium battery, a potassium-ion battery, a magnesium battery, a magnesium-ion battery, an aluminum battery, or an aluminum ion battery. Preferably, the battery is a lithium battery, a lithium-ion battery, sodium battery, a sodium-ion battery, a potassium battery, a potassium-ion battery, a magnesium battery, a magnesium-ion battery. In more preferred embodiments, the battery of the present invention is a lithium battery or lithium-ion battery, even more preferably a lithium-ion battery.

More details on the various components of the battery of the invention are provided in the following sections.

There is also provided a method of manufacturing and/or operating a battery as describe above, said method comprising the step of charging the battery up to an upper voltage limit of about 4.2 V or more, preferably about 4.4 V or more, about 4.6 V or more, about 4.8 V or more, about 5.0 V or more, about 5.2 V or more, about 5.4 V or more, or about 5.5 V or more.

### Carbonate Compound of Formula (I)

The carbonate compound of formula (I) is:

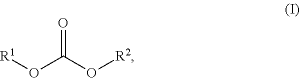

wherein
R¹ represents a C₃-C₂₄ alkyl, a C₃-C₂₄ alkoxyalkyl, a C₃-C₂₄ ω-O-alkyl oligo(ethylene glycol), or a C₄-C₂₄ ω-O-alkyl oligo(propylene glycol), and
R² represents a C₁-C₂₄ alkyl, a C₁-C₂₄ haloalkyl, a C₂-C₂₄ alkoxyalkyl, a C₂-C₂₄ alkyloyloxyalkyl, a C₃-C₂₄ alkoxycarbonylalkyl, a C₁-C₂₄ cyanoalkyl, a C₁-C₂₄ thiocyanatoalkyl, a C₃-C₂₄ trialkylsilyl, a C₄-C₂₄ trialkylsilylalkyl, a C₄-C₂₄ trialkylsilyloxyalkyl, a C₃-C₂₄ ω-O-alkyl oligo(ethylene glycol), a C₄-C₂₄ ω-O-alkyl oligo(propylene glycol), a C₅-C₂₄ ω-O-trialkylsilyl oligo(ethylene glycol), or a C₆-C₂₄ ω-O-trialkylsilyl oligo(propylene glycol).

In more preferred embodiments, R¹ represents a C₃-C₂₄ alkyl or a C₃-C₂₄ ω-O-alkyl oligo(ethylene glycol). In more preferred embodiments, R¹ represents a C₃-C₂₄ alkyl.

In more preferred embodiments, R² represents a C₁-C₂₄ alkyl, a C₂-C₂₄ alkoxyalkyl, a C₁-C₂₄ cyanoalkyl, a C₄-C₂₄ trialkylsilyloxyalkyl, a C₆-C₂₄ ω-O-trialkylsilyl oligo(ethylene glycol), or a C₃-C₂₄ ω-O-alkyl oligo(ethylene glycol). In most preferred embodiments, R² represents a C₁-C₂₄ alkyl.

Given that R¹ and R² as defined above contain at least 3 and 1 carbon atoms, respectively, the sum of the carbon atoms in R¹ and R² is at least 4. In preferred embodiments, the sum of the carbon atoms in R¹ and R² is:
5 or more, preferably 6 or more, more preferably 7 or more, yet more preferably 8 or more, and most preferably 9 or more, and/or
24 or less, preferably 20 or less, more preferably 16 or less, yet more preferably 14 or less, even more preferably 12 or less, and most preferably 10 or less.

Each of the alkyl and substituted alkyl in R¹ and R² are linear or branched.

Herein, “alkyl” has its usual meaning in the art. Specifically, it is a monovalent saturated aliphatic hydrocarbon radical of general formula —CₙH₂ₙ₊₁.

Non-limiting examples of C₃-C₂₄ alkyl in R¹ include propyl, isopropyl (2-propyl), butyl, 2-butyl, 3-butyl, isobutyl (3-methylpropyl), tertbutyl (2,2-dimethylethyl), 2-methylbutyl, 3-methylbutyl, 1-methyl-2-butyl, 2-methyl-2-butyl, 2-methylpentyl, 3-methylpentyl, 4-methylpentyl, 3-methyl-2-pentyl, 4-methyl-2-pentyl, 2-methyl-2-pentyl, 2-methyl-3-pentyl, 3-methyl-3-pentyl, 3,3-dimethyl-2-butyl, 2,3-dimethyl-2-butyl, 2-ethylbutyl, 3-ethyl-2-butyl, 2-ethylhexyl, pentyl and its isomers (including 2-pentyl and 3-pentyl), hexyl and its isomers (including 2-hexyl and 3-hexyl), heptyl and its isomers, octyl and its isomers, nonyl and its isomers, decyl and its isomers, undecyl and its isomers, and dodecyl and its isomers. In preferred embodiments, the C₃-C₂₄ alkyl in R¹ is a C₃-C₁₈ alkyl, preferably a C₃-C₁₂ alkyl, preferably a C₃-C₁₁ alkyl, preferably a C₃-C₁₀ alkyl, preferably a C₃-C₃ alkyl, more preferably a C₃-C₈ alkyl, even more preferably a C₃-C₇ alkyl (yet more preferably a C₄-C₇ alkyl), yet more preferably a C₃-C₆ alkyl (yet more preferably a C₄-C₆ alkyl), more preferably a C₃-C₅ alkyl, and most preferably a C₄-C₅ alkyl.

Non-limiting examples of C₁-C₂₄ alkyl chain in R² include the C₃-C₂₄ alkyls listed above with regard to R¹, as well as methyl and ethyl. In preferred embodiments, R² is a C₁-C₁₈ alkyl, preferably a C₁-C₁₂ alkyl, a C₁-C₉ alkyl, a C₁-C₈ alkyl, a C₁-C₇ alkyl, a C₄-C₇ alkyl, a C₃-C₇ alkyl (preferably a C₄-C₇ alkyl), a C₃-C₆ alkyl (preferably a C₄-C₆ alkyl), a C₃-C₅ alkyl, and most preferably a C₄-C₅ alkyl.

In preferred embodiments, both R¹ and R² are alkyl groups. In more preferred embodiments, R¹ and R² are the same alkyl groups. In alternative preferred embodiments, R¹ and R² are different alkyl groups.

Preferred C₃ alkyls in R¹ and R² include propyl, and isopropyl (2-propyl).

Preferred C₄ alkyls in R¹ and R² include butyl, 2-butyl, 3-butyl, isobutyl (3-methylpropyl), and tertbutyl (2,2-dimethylethyl).

Preferred C₅ alkyls in R¹ and R² include pentyl and its isomers (including 2-pentyl and 3-pentyl), 2-methylbutyl, 3-methylbutyl, 1-methyl-2-butyl, and 2-methyl-2-butyl.

Preferred C₅ alkyls in R¹ and R² include hexyl and its isomers (including 2-hexyl and 3-hexyl), 2-methylpentyl, 3-methylpentyl, 4-methylpentyl, 3-methyl-2-pentyl, 4-methyl-2-pentyl, 2-methyl-2-pentyl, 2-methyl-3-pentyl, 3-methyl-3-pentyl, 3,3-dimethyl-2-butyl, 2,3-dimethyl-2-butyl, 2-ethylbutyl, and 3-ethyl-2-butyl.

Preferred C₇ alkyls in R¹ and R² include heptyl and its isomers.

Preferred C₈ alkyls in R¹ and R² include 2-ethylhexyl.

Herein, a “haloalkyl” refers to an alkyl group in which one or more (or even all) of the hydrogen atoms are each replaced by a halogen atom, wherein the halogen atoms are the same or different from one another (when more than one halogen atoms are present). Halogen atoms include fluorine (F), chlorine (Cl), bromine (Br), and iodine (I). Preferably, the halogen atom is fluorine. Non-limiting examples of C₁-C₂₄ haloalkyls in R² include trifluoromethyl, pentafluoroethyl, heptafluoropropyl, nonafluorobutyl, 2,2,2-trifluoroethyl, and 1,1,1,3,3,3-hexafluoro-2-propyl.

Herein, an “alkoxyalkyl” refers to an alkyl group in which one or more, preferably one, of the hydrogen atoms are each replaced by an alkoxy group, wherein the alkoxy groups are the same or different from one another (when more than one alkoxy groups are present). In preferred embodiments, the alkoxyalkyl comprises only one alkoxy group. An alkoxy group is a radical of formula —O-alkyl, this alkyl being linear or branched, preferably linear. A C₂-C₂₄ alkoxyalkyl is alkoxyalkyl radical, wherein the sum of the number of carbon atoms contained in the alkyl and alkoxy groups is between 2 and 24. In preferred embodiments, the alkoxyalkyl is a (C₁-C₂)alkoxy(C₂-C₅)alkyl. Non-limiting examples of alkoxyalkyls in R² or R¹ include 2-methoxyethyl, 3-methoxypropyl, 2-methoxypropyl, 4-methoxybutyl, 4-ethoxybutyl, 5-methoxypentyl, 6-methoxyhexyl, and 2-isopropoxyethyl. In preferred embodiments, the alkoxyalkyl is 2-methoxyethyl or 2-isopropoxyethyl.

Herein, an “alkyloyloxyalkyl” refers to an alkyl group in which one or more, preferably one, of the hydrogen atoms are each replaced by an alkyloyloxy group, wherein the alkyloyloxy groups are the same or different when more than one alkyloyloxy groups are present). In preferred embodiments, the alkyloyloxyalkyl comprises only one alkyloyloxy group. An alkyloyloxy group is a radical of formula —O—C(═O)-alkyl, this alkyl being linear or branched. A C₂-C₂₄ alkyloyloxyalkyl is alkyloyloxyalkyl wherein the sum of the number of carbon atoms contained in the alkyl and alkyloyloxy groups is between 2 and 24. Non-limiting examples of C₂-C₂₄ alkyloyloxyalkyl in R² include 2-acetoxyethyl, 3-acetoxypropyl, 2-acetoxypropyl, and 4-acetoxybutyl.

Herein, an “alkoxycarbonylalkyl” refers to an alkyl group in which one or more of the hydrogen atoms are each replaced by an alkoxycarbonyl group, wherein the alkoxycarbonyl groups are the same or different from one another (when more than one alkoxycarbonyl groups are present). In preferred embodiments, the alkoxycarbonylalkyl comprises only one alkoxycarbonyl group. An alkoxycarbonyl group is a radical of formula C(═O)—O-alkyl, this alkyl being linear or branched. A C₂-C₂₄ alkoxycarbonyl is an alkoxycarbonyl wherein the sum of the number of carbon atoms contained in the alkyl and alkoxycarbonyl groups is between 3 and 24. Non-limiting examples of C₃-C₂₄ alkoxycarbonylalkyl in R² include 2-ethoxycarbonylethyl and 3-methoxycarbonylpropyl.

Herein, a “cyanoalkyl” refers to an alkyl group in which one or more of the hydrogen atoms are each replaced by a cyano (—C≡N) group. In preferred embodiments, the cyanoalkyl comprises only one cyano group. In preferred embodiments, the cyanoalkyl is a C₁-C₅ cyanoalkyl. Non-limiting examples of C₁-C₂₄ cyanoalkyls in R² include cyanomethyl, 2-cyanoethyl, 3-cyanopropyl, 4-cyanobutyl, and 5-cyanopentyl. In preferred embodiments, the C₁-C₂₄ cyanoalkyl in R² is 2-cyanoethyl.

Herein, a “thiocyanatoalkyl” refers to an alkyl group in which one or more of the hydrogen atoms are each replaced by a thiocyanato (—S—C≡N) group. In preferred embodiments, the thiocyanatoalkyl comprises only one thiocyanato group. Non-limiting examples of C₁-C₂₄ thiocyanatoalkyls in R² include thiocyanatomethyl, 2-thiocyanatoethyl, 3-thiocyanatopropyl, 4-thiocyanatobutyl, 5-thiocyanatopentyl, and 6-thiocyanatohexyl.

Herein, a “trialkylsilyl” refers to a radical of formula (alkyl)₃-Si—, wherein the alkyl groups are the same or different and are linear or brandied. A C₃-C₂₄ trialkylsilyl is a trialkylsilyl wherein the sum of the number of carbon atoms contained in all of the alkyl groups is between 3 and 24. In preferred embodiments, each of the alkyl groups in the trialkylsilyl is a C₁-C₄ alkyl group. In preferred embodiments, the three alkyl groups are the same. Non-limiting examples of C₃-C₂₄ trialkylsilyls in R² include trimethylsilyl, ethyldimethylsilyl, diethylmethylsilyl, triethylsilyl, dimethylpropylsilyl, dimethylisopropylsilyl, triisopropylsilyl, butyldimethylsilyl, and tertbutyldimethylsilyl.

Herein, a “trialkylsilylalkyl” is an alkyl group in which one or more of the hydrogen atoms are each replaced by a trialkylsilyl group, wherein the trialkylsilyl are as defined above and are the same or different from one another (when more than one trialkylsilyl groups ae present). In a C₄-C₂₄ trialkylsilylalkyl, the sum of the number of carbon atoms contained in all four of the alkyl groups is between 4 and 24. Preferably, the trialkylsilylalkyl comprises only one trialkylsilyl group. In preferred embodiments, the C₄-C₂₄ trialkylsilylalkyl is a trialkylsilylalkyl(C₁-C₄)alkyl, preferably a trialkylsilylalkyl(C₂-C₄)alkyl. In preferred embodiments, the three alkyl groups attached to the Si atom are methyl groups. Non-limiting examples of C₄-C₂₄ trialkylsilylalkyl in R² include trimethylsilylethyl, 2-trimethylsilylethyl, 3-trimethylsilylpropyl and 4-trimethylsilylbutyl.

Herein, a “trialkylsiyloxyalkyl” refers to an alkyl group in which one or more of the hydrogen atoms are each replaced by a trialkylsilyloxy group, wherein the trialkylsilyloxy groups are the same or different from one another (when more than one trialkylsilyloxy groups are present). Preferably, the trialkylsilyloxyalkyl comprises only one trialkylsilyloxy group. Herein, a “trialkylsilyloxy” is a radical of formula (alkyl)₃-Si—O—, wherein the alkyl groups are the same or different from one another and are linear or branched. In a C₄-C₂₄ trialkylsilyloxyalkyl, the sum of the number of carbon atoms contained in all four of the alkyl groups is between 4 and 24. In preferred embodiments, the C₄-C₂₄ trialkylsilyloxyalkyl is a trialkylsilyloxy(C₃-C₄)alkyl. In preferred embodiments, the three alkyl groups attached to the Si atom are methyl groups. Non-limiting examples of C₄-C₂₄ trialkylsilyloxyalkyl in R² include (2-trimethylsilyloxy)ethyl, 3-trimethylsilyloxypropyl and 4-trimethylsilyloxybutyl. In preferred embodiments, the C₄-C₂₄ trialkylsilyloxyalkyl in R² is (2-trimethylsilyloxy)ethyl.

Herein an ω-O-alkyl oligo(ethylene glycol) is a radical of formula —(CH₂—CH₂—O—)ₙ-alkyl, wherein n is 1 or more. In a C₃-C₂₄ ω-O-alkyl oligo(ethylene glycol), the sum of the number of carbon atoms contained in the alkyl and the (CH₂—CH₂—O—) repeating motif(s) is between 3 and 24. In preferred embodiments, n is an integer from 1 to 5. In preferred embodiments, the alkyl group is a C₁-C₄ alkyl. Non-limiting examples of ω-O-alkyl oligo(ethylene glycol) in R² or R¹ include 2-methoxyethyl, 2-ethoxyethyl, 2-propoxyethyl, 2-isopropoxyethyl, 2-butyloxyethyl, 2-(2-methoxyethoxy)ethyl, 2-(2-butoxyethoxy)ethyl, 2-(2-ethoxyethoxy)ethyl, 2-[2-(2-methoxyethoxy)ethoxy]ethyl, 2-[2-(2-ethoxyethoxy)ethoxy]ethyl, 2,5,8,11-tetraoxatridecyl, 3,6,9,12-tetraoxatetradecyl, 2,5,8,11,14-pentaoxahexadecyl, or 3,6,9,12,15-pentaoxaheptadecyl. In preferred embodiments, the ω-O-alkyl oligo(ethylene glycol) of R² or R¹ is 2-methoxyethyl, 2-isopropoxyethyl, or 2-(2-methoxyethoxy)ethyl.

Herein an ω-O-alkyl oligo(propylene glycol) is a radical of formula —(CH₂—CH₂—CH₂—O—)ₙ-alkyl, wherein n is 1 or more. In a C₄-C₂₄ ω-O-alkyl oligo(propylene glycol), the sum of the number of carbon atoms contained in the alkyl and the (CH₂—CH₂—CH₂—O—) repeating motif(s) is between 4 and 24. In preferred embodiments, n is 1. In preferred embodiments, the alkyl group is a C₁-C₄ alkyl. Non-limiting examples of ω-O-alkyl oligo(propylene glycol) in R² or R¹ include 2-methoxypropyl, 2-ethoxypropyl, 1-methoxy-2-propyl, 1-ethoxy-2-propyl, 1-propoxy-2-propyl, 1-isopropoxy-2-propyl, and 1-butoxy-2-propyl.

Herein an ω-O-trialkylsilyl oligo(ethylene glycol) is a radical of formula —(CH₂—CH₂—O—)ₙ—Si-(alkyl)₃, wherein the alkyl groups are the same or different and are linear or branched and wherein n is 1 or more. In a C₅-C₂₄ ω-O-trialkylsilyl oligo(ethylene glycol), the sum of the number of carbon atoms contained in the alkyl groups and the (CH₂—CH₂—O—) repeating motif(s) is between 5 and 24. In preferred embodiments, n is an integer from 1 to 5. In preferred embodiments, the three alkyl groups (attached to the Si atom) are methyl groups. Non-limiting examples of ω-O-trialkylsilyl oligo(ethylene glycol) in R² include 2-trimethylsilyloxyethyl, 2-(2-trimethylsilyloxyethoxy)ethyl, 2-[2-(2-trimethylsilyloxyethoxy)-ethoxy]ethyl, 2-{2-[2-(2-trimethylsilyloxyethoxy)ethoxy]ethoxy}ethyl, and 2-(2-{2-[2-(2-trimethylsilyloxyethoxy)ethoxy]ethoxy}ethoxy)ethyl. In preferred embodiments, the ω-O-trialkylsiyl oligo(ethylene glycol) of R² is 2-trimethylsilyloxyethyl.

Herein an ω-O-trialkylsilyl oligo(propylene glycol) is a radical of formula —(CH₂—CH₂—CH₂—O—)ₙ—Si-(alkyl)₃, wherein the alkyl groups are the same or different and are linear or branched and wherein n is 1 or more. In a C₆-C₂₄ ω-O-trialkylsilyl oligo(ethylene glycol), the sum of the number of carbon atoms contained in the alkyl groups and the (CH₂—CH₂—CH₂—O—) repeating motif(s) is between 6 and 24. In preferred embodiments, n is 1. In preferred embodiments, the three alkyl groups (attached to the Si atom) are methyl groups. Non-limiting examples of ω-O-trialkylsilyl oligo(propylene glycol) in R² include 2-trimethylsiyloxypropyl, and 1-trimethylsilyloxy-2-propyl.

Preferably, the carbonate compound of formula (I) is: isopropyl methyl carbonate, methyl propyl carbonate, ethyl propyl carbonate, ethyl isopropyl carbonate, dipropyl carbonate, isopropyl propyl carbonate, diisopropyl carbonate, butyl methyl carbonate, butyl ethyl carbonate, butyl propyl carbonate, dibutyl carbonate, butyl isopropyl carbonate, 2-butyl methyl carbonate, 2-butyl ethyl carbonate, 2-butyl propyl carbonate, di(2-butyl) carbonate, 2-butyl isopropyl carbonate, isobutyl methyl carbonate, isobutyl ethyl carbonate, isobutyl propyl carbonate, diisobutyl carbonate, isobutyl isopropyl carbonate, 2-butyl isobutyl carbonate, 2-ethylbutyl methyl carbonate, di(2-ethylbutyl) carbonate, methyl pentyl carbonate, ethyl pentyl carbonate, pentyl propyl carbonate, butyl pentyl carbonate, dipentyl carbonate, isopropyl pentyl carbonate, 2-butyl pentyl carbonate, isobutyl pentyl carbonate, methyl 2-pentyl carbonate, ethyl 2-pentyl carbonate, 2-pentyl propyl carbonate, butyl 2-pentyl carbonate, di(2-pentyl) carbonate, isopropyl 2-pentyl carbonate, 2-butyl 2-pentyl carbonate, isobutyl 2-pentyl carbonate, methyl 3-pentyl carbonate, ethyl 3-pentyl carbonate, 3-pentyl propyl carbonate, butyl 3-pentyl carbonate, di(3-pentyl) carbonate, isopropyl 3-pentyl carbonate, 2-butyl 3-pentyl carbonate, isobutyl 3-pentyl carbonate, pentyl 2-pentyl carbonate, pentyl 3-pentyl carbonate, 2-pentyl 3-pentyl carbonate, methyl hexyl carbonate, ethyl hexyl carbonate, propyl hexyl carbonate, butyl hexyl carbonate, pentyl hexyl carbonate, dihexyl carbonate, isopropyl hexyl carbonate, isobutyl hexyl carbonate, di(2-ethylhexyl) carbonate, 2-ethylhexyl methyl carbonate, didodecyl carbonate, ethyl dodecyl carbonate, cyanomethyl propyl carbonate, butyl cyanomethyl carbonate, cyanomethyl isopropyl carbonate, 2-butyl cyanomethyl carbonate, isobutyl cyanomethyl carbonate, tertbutyl cyanomethyl carbonate, cyanomethyl pentyl carbonate, cyanomethyl 2-pentyl carbonate, cyanomethyl 3-pentyl carbonate, cyanomethyl hexyl carbonate, cyanomethyl heptyl carbonate, cyanomethyl octyl carbonate, cyanomethyl nonyl carbonate, cyanomethyl decyl carbonate, cyanomethyl undecyl carbonate, cyanomethyl dodecyl carbonate, cyanomethyl 2-ethylhexyl carbonate, 2-cyanoethyl propyl carbonate, butyl 2-cyanoethyl carbonate, 2-cyanoethyl isopropyl carbonate, 2-butyl 2-cyanoethyl carbonate, isobutyl 2-cyanoethyl carbonate, tertbutyl 2-cyanoethyl carbonate, 2-cyanoethyl pentyl carbonate, 2-cyanoethyl 2-pentyl carbonate, 2-cyanoethyl 3-pentyl carbonate, 2-cyanoethyl hexyl carbonate, 2-cyanoethyl heptyl carbonate, 2-cyanoethyl octyl carbonate, 2-cyanoethyl nonyl carbonate, 2-cyanoethyl decyl carbonate, 2-cyanoethyl undecyl carbonate, 2-cyanoethyl dodecyl carbonate, 2-cyanoethyl 2-ethylhexyl carbonate, 3-cyanopropyl propyl carbonate, butyl 3-cyanopropyl carbonate, 3-cyanopropyl isopropyl carbonate, 2-butyl 3-cyanopropyl carbonate, isobutyl 3-cyanopropyl carbonate, tertbutyl 3-cyanopropyl carbonate, 3-cyanopropyl pentyl carbonate, 3-cyanopropyl 2-pentyl carbonate, 3-cyanopropyl 3-pentyl carbonate, 3-cyanopropyl hexyl carbonate, 3-cyanopropyl heptyl carbonate, 3-cyanopropyl octyl carbonate, 3-cyanopropyl nonyl carbonate, 3-cyanopropyl decyl carbonate, 3-cyanopropyl undecyl carbonate, 3-cyanopropyl dodecyl carbonate, 3-cyanopropyl 2-ethylhexyl carbonate, 4-cyanobutyl propyl carbonate, butyl 4-cyanobutyl carbonate, 4-cyanobutyl isopropyl carbonate, 2-butyl 4-cyanobutyl carbonate, isobutyl 4-cyanobutyl carbonate, tertbutyl 4-cyanobutyl carbonate, 4-cyanobutyl pentyl carbonate, 4-cyanobutyl 2-pentyl carbonate, 4-cyanobutyl 3-pentyl carbonate, 4-cyanobutyl hexyl carbonate, 4-cyanobutyl heptyl carbonate, 4-cyanobutyl octyl carbonate, 4-cyanobutyl nonyl carbonate, 4-cyanobutyl decyl carbonate, 4-cyanobutyl undecyl carbonate, 4-cyanobutyl dodecyl carbonate, 4-cyanobutyl 2-ethylhexyl carbonate, propyl trimethylsilyl carbonate, butyl trimethylsilyl carbonate, isopropyl trimethylsilyl carbonate, 2-butyl trimethylsilyl carbonate, isobutyl trimethylsilyl carbonate, tertbutyl trimethylsilyl carbonate, pentyl trimethylsilyl carbonate, 2-pentyl trimethylsilyl carbonate, 3-pentyl trimethylsilyl carbonate, hexyl trimethylsilyl carbonate, heptyl trimethylsilyl carbonate, octyl trimethylsilyl carbonate, nonyl trimethylsilyl carbonate, decyl trimethylsilyl carbonate, trimethylsilyl undecyl carbonate, dodecyl trimethylsilyl carbonate, 2-ethylhexyl trimethylsilyl carbonate, ethyldimethylsilyl propyl carbonate, butyl ethyldimethylsilyl carbonate, ethyldimethylsilyl isopropyl carbonate, 2-butyl ethyldimethylsilyl carbonate, isobutyl ethyldimethylsilyl carbonate, tertbutyl ethyldimethylsilyl carbonate, ethyldimethylsilyl pentyl carbonate, ethyldimethylsilyl 2-pentyl carbonate, ethyldimethylsilyl 3-pentyl carbonate, ethyldimethylsilyl hexyl carbonate, ethyldimethylsilyl heptyl carbonate, ethyldimethylsilyl octyl carbonate, ethyldimethylsilyl nonyl carbonate, decyl ethyldimethylsilyl carbonate, ethyldimethylsilyl undecyl carbonate, dodecyl ethyldimethylsilyl carbonate, ethyldimethylsilyl 2-ethylhexyl carbonate, diethylmethylsilyl propyl carbonate, butyl diethylmethylsilyl carbonate, diethylmethylsilyl isopropyl carbonate, 2-butyl diethylmethylsilyl carbonate, isobutyl diethylmethylsilyl carbonate, tertbutyl diethylmethylsilyl carbonate, diethylmethylsilyl pentyl carbonate, diethylmethylsilyl 2-pentyl carbonate, diethylmethylsilyl 3-pentyl carbonate, diethylmethylsilyl hexyl carbonate, diethylmethylsilyl heptyl carbonate, diethylmethylsilyl octyl carbonate, diethyimethylsilyl nonyl carbonate, decyl diethylmethylsilyl carbonate, diethylmethylsilyl undecyl carbonate, diethylmethylsilyl dodecyl carbonate, 2-ethylhexyl diethylmethylsilyl carbonate, propyl triethylsilyl carbonate, butyl triethylsilyl carbonate, isopropyl triethylsilyl carbonate, 2-butyl triethylsilyl carbonate, isobutyl triethylsilyl carbonate, tertbutyl triethylsilyl carbonate, pentyl triethylsilyl carbonate, 2-pentyl triethylsilyl carbonate, 3-pentyl triethylsilyl carbonate, hexyl triethylsilyl carbonate, heptyl triethylsilyl carbonate, octyl triethylsilyl carbonate, nonyl triethylsilyl carbonate, decyl triethylsilyl carbonate, triethylsilyl undecyl carbonate, dodecyl triethylsilyl carbonate, 2-ethyihexyl triethylsilyl carbonate, dimethylisopropylsilyl propyl carbonate, butyl dimethylisopropylsilyl carbonate, dimethylisopropylsilyl isopropyl carbonate, 2-butyl dimethylisopropylsilyl carbonate, isobutyl dimethylisopropylsilyl carbonate, tertbutyl dimethylisopropylsilyl carbonate, dimethylisopropylsilyl pentyl carbonate, dimethylisopropylsilyl 2-pentyl carbonate, dimethylisopropylsilyl 3-pentyl carbonate, dimethylisopropylsilyl hexyl carbonate, dimethylisopropylsilyl heptyl carbonate, dimethylisopropylsilyl octyl carbonate, dimethylisopropylsilyl nonyl carbonate, decyl dimethylisopropylsilyl carbonate, dimethylisopropylsilyl undecyl carbonate, dimethylisopropylsilyl dodecyl carbonate, 2-ethylhexyl dimethylisopropylsilyl carbonate, propyl triisopropylsilyl carbonate, butyl triisopropylsilyl carbonate, isopropyl triisopropylsilyl carbonate, 2-butyl triisopropylsilyl carbonate, isobutyl triisopropylsilyl carbonate, tertbutyl triisopropylsilyl carbonate, pentyl triisopropylsilyl carbonate, 2-pentyl triisopropylsilyl carbonate, 3-pentyl triisopropylsilyl carbonate, hexyl triisopropylsilyl carbonate, heptyl triisopropylsilyl carbonate, octyl triisopropylsilyl carbonate, nonyl triisopropylsilyl carbonate, decyl triisopropylsilyl carbonate, triisopropylsilyl undecyl carbonate, dodecyl triisopropylsilyl carbonate, 2-ethylhexyl triisopropylsilyl carbonate, propyl tertbutyldimethylsilyl carbonate, butyl tertbutyldimethylsilyl carbonate, isopropyl tertbutyldimethylsilyl carbonate, 2-butyl tertbutyldimethylsilyl carbonate, isobutyl tertbutyldimethylsilyl carbonate, tertbutyl tertbutyldimethylsilyl carbonate, pentyl tertbutyldimethylsilyl carbonate, 2-pentyl tertbutyldimethylsilyl carbonate, 3-pentyl tertbutyldimethylsilyl carbonate, hexyl tertbutyldimethylsilyl carbonate, heptyl tertbutyldimethylsilyl carbonate, octyl tertbutyldimethylsilyl carbonate, nonyl tertbutyldimethylsilyl carbonate, decyl tertbutyldimethylsilyl carbonate, tertbutyldimethylsilyl undecyl carbonate, dodecyl tertbutyldimethylsilyl carbonate, 2-ethylhexyl tertbutyldimethylsilyl carbonate, propyl 2-trimethylsilylethyl carbonate, butyl 2-trimethylsilylethyl carbonate, isopropyl 2-trimethylsilylethyl carbonate, 2-butyl 2-trimethylsilylethyl carbonate, isobutyl 2-trimethylsilylethyl carbonate, tertbutyl 2-trimethylsilylethyl carbonate, pentyl 2-trimethylsilylethyl carbonate, 2-pentyl 2-trimethylsilylethyl carbonate, 3-pentyl 2-trimethylsilylethyl carbonate, hexyl 2-trimethylsilylethyl carbonate, heptyl 2-trimethylsilylethyl carbonate, octyl 2-trimethylsilylethyl carbonate, nonyl 2-trimethylsilylethyl carbonate, decyl 2-trimethylsilylethyl carbonate, 2-trimethylsilylethyl undecyl carbonate, dodecyl 2-trimethylsilylethyl carbonate, 2-ethylhexyl 2-trimethylsilylethyl carbonate, 2-methoxyethyl isobutyl carbonate, or (2-trimethylsilyloxy)ethyl butyl carbonate.

In more preferred embodiments, the carbonate compound of formula (I) is didodecyl carbonate, dibutyl carbonate, dipropyl carbonate, methyl propyl carbonate, diisopropyl carbonate, isopropyl methyl carbonate, ethyl dodecyl carbonate, ethyl propyl carbonate, ethyl isopropyl carbonate, diisobutyl carbonate, isobutyl methyl carbonate, dipentyl carbonate, methyl pentyl carbonate, di(2-ethylhexyl) carbonate, 2-ethylhexyl methyl carbonate, methyl 2-pentyl carbonate, di(2-pentyl) carbonate, 2-butyl methyl carbonate, di(2-butyl) carbonate, 2-ethylbutyl methyl carbonate, di(2-ethylbutyl) carbonate, isobutyl isopropyl carbonate, 2-cyanoethyl butyl carbonate, 2-methoxyethyl isobutyl carbonate, (2-trimethylsilyloxy)ethyl butyl carbonate, di(2-methoxyethyl) carbonate, 2-isopropoxyethyl methyl carbonate, di(2-isopropoxyethyl) carbonate, or di(2-(2-methoxyethoxy)ethyl) carbonate.

In more preferred embodiments, the compound of formula (I) is didodecyl carbonate, dibutyl carbonate, 2-ethylbutyl methyl carbonate, di(2-ethylbutyl) carbonate, di(2-butyl) carbonate, di(2-ethylhexyl) carbonate, 2-ethylhexyl methyl carbonate, di(2-pentyl) carbonate, ethyl dodecyl carbonate, 2-cyanoethyl butyl carbonate, 2-methoxyethyl isobutyl carbonate, (2-trimethylsilyloxy)ethyl butyl carbonate, di(2-isopropoxyethyl) carbonate, or diisobutyl carbonate.

In even more preferred embodiments, the compound of formula (I) is didodecyl carbonate, di(2-ethylhexyl) carbonate, 2-ethylhexyl methyl carbonate, ethyl dodecyl carbonate, or diisobutyl carbonate.

In a most preferred embodiment, the carbonate compound of formula (I) is diisobutyl carbonate.

In another aspect of the invention, the invention provides all of the above carbonate compounds per se, including all preferred subgroups thereof, especially those wherein, when R² is a C₁-C₉ alkyl, R¹ is not a C₃-C₉ alkyl. Preferred such compounds include those in which, when R² is a C₁-C₉ alkyl, R¹ represents a C₃-C₂₄ alkoxyalkyl, a C₃-C₂₄ ω-O-alkyl oligo(ethylene glycol), or a C₄-C₂₄ ω-O-alkyl oligo(propylene glycol).

### Non-Autocue Electrolyte

As noted above, the low-corrosiveness non-aqueous electrolyte comprises, as a solvent, the carbonate compound of formula (I) of the previous section as well as a conducting salt dissolved in said solvent.

In embodiments, mixtures of said carbonate compounds of formula (I) may be used as said solvent.

The electrolyte of the present invention can be prepared using any known technique in the art. For example, to prepare electrolytes from the carbonate solvents of the present invention, the skilled person would know that an appropriate conducting salt can be dissolved in said carbonate solvents in an appropriate concentration. Depending on the application of the electrolyte, a different salt can be chosen. For example, as described above, a lithium salt can be chosen when the electrolyte will be used in a lithium battery. However, for sodium, potassium, calcium, aluminum and magnesium based batteries, other salts can be dissolved in the solvents, for example sodium, potassium, calcium, aluminum and magnesium salts.

### Conducting Salt

The voice of the conducting salt has an impact on anodic dissolution. For example, for an electrolyte containing less of the carbonate compound of formula (I) of the present invention, the addition of a passivating conducting salt will produce an electrolyte which nonetheless prevents anodic dissolution of aluminum. Some inorganic salts like LiPF6 passivate the surface of the aluminum, as they form insoluble compounds and thus do not cause anodic dissolution up to more than 5 V vs Li anodes. In contrast, some salts do not passivate aluminum, especially lower fluorinated sulfonyl amides, which cause a very strong dissolution of aluminum. As mentioned, this can lead to malfunctioning of the battery system if its operating voltage surpasses the critical potential. When such conducting salts are used, it is preferable to include more of the carbonate compound of formula (I) in the electrolyte so as to further prevent anodic dissolution.

The conducting salt can be chosen from: LiClO₄; LiP(CN)_{α}F_{6-α}, where α is an integer from 0 to 6, preferably LiPF₆; LiB(CN)_{β}F_{4-β}, where β is an integer from 0 to 4, preferably LiBF₄; LiP(CₙF₂ₙ₊₁)_{γ}F_{6_γ}, where n is an integer from 1 to 20, and γ is an integer from 1 to 6; LiB(CₙF₂ₙ₊₁)_{δ}F_{4-δ}, where n is an integer from 1 to 20, and δ is an integer from 1 to 4; Li₂Si(CₙF₂ₙ₊₁)_{ε}F_{6-ε}, where n is an integer from 1 to 20, and ε is an integer from 0 to 6; lithium bisoxalato borate; lithium difluorooxalatoborate; and compounds represented by the following general formulas:

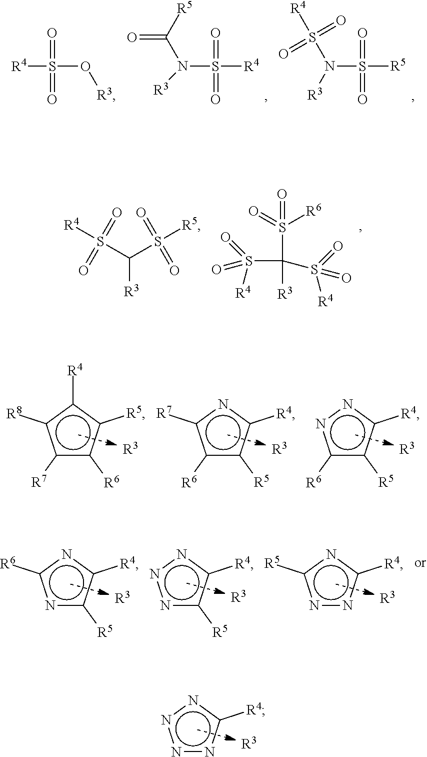

wherein
R³ represents: Li, Na, K, Rb, Cs, Be, Mg, Ca, Sr, Ba, Al, hydrogen, or an organic cation; and
R⁴, R⁵, R⁶, R⁷, R⁸ represent cyano, fluorine, chlorine, branched or linear alkyl radical with 1-24 carbon atoms, perfluorinated linear alkyl radical with 1-24 carbon atoms, aryl or heteroaryl radical, or perfluorinated aryl or heteroaryl radical;
and their derivatives.

In preferred embodiments, the conducting salt is a lithium salt. This is appropriate when, for example, the electrolyte will be used in a lithium or lithium-ion battery. Non-limiting examples of lithium salts include the above salts, preferably lithium perchlorate, lithium tetrafluoroborate, lithium hexafluorophosphate, lithium sulfonyl amide salts (such as lithium bis(fluorosulfonyl)amide, lithium N-fluorosulfonyl-trifluoromethanesulfonyl amide (LiFTFSI), and lithium bis(trifluoromethanesulfonyl)amide) and their derivatives. In preferred embodiments, the conducting salt is a lithium sulfonylamide salt. In preferred embodiments, the lithium sulfonyl amide salt is lithium bis(fluorosulfonyl)amide (LiFSI), lithium bis(trifluoromethanesulfonyl)amide (LiTFSI), or lithium N-flurosulfonyl-trifluoromethanesulfonyl amide (LiFTFSI). In more preferred embodiments, the conducting salt is LiFSI. This is appropriate when, for example, the electrolyte is to be used in a lithium-ion battery. Indeed, an important advantage of the electrolyte of the present invention is that it enables use of lithium sulfonylamide salts in battery systems where the upper potential limit of the cathode is above 4.2 V vs Li metal.

In alternative embodiments, the salt is a sodium, a potassium, calcium, aluminum, or a magnesium salt such as those listed above. This is appropriate when, for example, the electrolyte is to be used in a sodium-, potassium-, calcium-, aluminum-, or magnesium-based battery.

The concentration of the conducting salt present in the electrolyte may vary; the skilled person would understand that the quantity of conducting salt should not severely negatively impact the efficacy of the electrolyte. The concentration of the conducting salt refers to the molarity of the conducting salt in the carbonate solvent and any other solvents (if present), disregarding the presence of additives. This can be represented by the following equation:

$$Concentration of conducting salt = \frac{moles of conducting salt}{volume of the electrolyte}$$

wherein the volume of the electrolyte is the final total volume of the carbonate compound of formula (I), the dissolved salt, and any liquid additive present

In embodiments, the concentration of the conducting salt is at least about 0.05 M and/or at most about 3 M. In embodiments, the concentration of the conducting salt is at least about 0.05 M, at least about 0.1 M, at least about 0.5 M, or at least about 1 M, and/or at most about 3 M, at most about 2 M, at most about 1.5 M, or at most about 1 M.

In preferred embodiments, the concentration of the conducting salt is 1 M.

Additives that Improve the Electrochemical Properties of the Electrolyte

In embodiments, the electrolyte further comprises one or more additives, which are used to improve the electrochemical properties of the electrolyte. Non-limiting examples of additives that improve the electrochemical properties of the electrolyte include:
agents that improve solid electrolyte interphase (SEI) and cycling properties,
unsaturated carbonates that improve stability at high and low voltages, and
organic solvents that diminish viscosity and increase conductivity.

It will be understood by the skilled person that one additive can have more than one specific technical effect on the electrolyte and thus may be cited in more than one of the above lists of exemplary additives with different preferred concentration ranges according to the effect desired of the additive.

Agents that improve solid electrolyte interphase and cycling properties are preferably present in the electrolyte. Non-limiting examples of agents that improve solid electrolyte interphase and cycling properties include ethylene carbonate, vinylene carbonate, fluorovinylene carbonate, succinic anhydride, malefic anhydride, fluoroethylene carbonate, difluoroethylene carbonate, methylene-ethylene carbonate, prop-1-ene-1,3-sultone, acrylamide, fumaronitrile, and triallyl phosphate. Preferred agents that improve solid electrolyte interphase and cycling properties include ethylene carbonate (EC) and fluoroethylene carbonate (FEC).

Unsaturated carbonates are optionally present in the electrolyte. Non-limiting examples of unsaturated carbonates that improve stability at high and low voltages include vinylene carbonate and derivatives of ethene (that is, vinyl compounds) like methyl vinyl carbonate, divinylcarbonate, and ethyl vinyl carbonate.

Organic solvents that diminish viscosity and increase conductivity are optionally present in the electrolyte. In preferred embodiments, such organic solvents are present Non-limiting examples of organic solvents that diminish viscosity and increase conductivity include polar solvents, preferably alkyl carbonates, alkyl ethers, and alkyl esters. For example, the organic solvent may be ethylene carbonate, propylene carbonate, dimethyl carbonate, ethyl methyl carbonate, diethyl carbonate, dimethoxyethane, diglyme (diethylene glycol dimethyl ether), triglyme (triethylene glycol dimethyl ether), tetraglyme ((tetraethylene glycol dimethyl ether), tetrahydrofuran, 2-methyltetrahydrofuran, 1,3-dioxolane, 2,2-dimethyl-1,3-dioxolane, 1,4-dioxane, 1,3-dioxane, methoxypropionitril, propionitril, butyronitrile, succinonitrile, glutaronitrile, adiponitrile, esters of acetic acid, esters of propionic acid, cyclic esters like γ-butyrolactone, ε-caprolactone, esters of trifluoroacetic acid, sulfolane, dimethyl sulfone, ethyl methyl sulfone, or peralkylated sulfamides. In embodiments, ionic liquids could also be added in order to diminish flammability and to increase conductivity. Preferred organic solvents that diminish viscosity and increase conductivity include ethylene carbonate (EC), dimethyl carbonate (DMC), ethyl methyl carbonate (EMC) and diethyl carbonate (DEC).

In embodiments, the agent(s) that improve solid electrolyte interphase (SEI) and cycling properties and the unsaturated carbonate(s), taken together, represents a total of at least about 0.1% and/or at most about 20% of the total mass of the electrolyte. In embodiments, the amount of these additives represents a total of at least about 0.1% w/w, at least 1% w/w, at least about 2% w/w, at least about 5% w/w, or at least about 7% w/w, and/or at most about 20% w/w, at most about 15% w/w, at most about 10% w/w, or at most about 7% w/w of the total weight of the electrolyte.

In embodiments, the organic solvents that diminish viscosity and increase conductivity represents a total of at least about 1% v/v and/or at most about 80% v/v of the total volume of the electrolyte. In embodiments, the f organic solvents represents a total of at least about 1% v/v, at least about 2% v/v, at least about 5% v/v, or at least about 7% v/v, and/or at most about 80% v/v, at most about 50% v/v, at most about 20% v/v, at most about 15% v/v, at most about 10% v/v, or at most about 7% v/v of the total volume of the electrolyte.

In preferred embodiments, the additives are fluoroethylene carbonate (FEC), ethylene carbonate (EC), diethyl carbonate (DEC), or a mixture thereof.

In more preferred embodiments, the additives are FEC, preferably about 2 w/w % of FEC, alone or together with EC, DEC or a mixture thereof, preferably alone or together with:
up to about 5% v/v of EC,
up to about 10% v/v of EC,
up to about 15% v/v of EC,
up to about 20% v/v of EC,
up to about 30% v/v of EC,
up to about 20% v/v of a mixture of EC and DEC,
up to about 25% v/v of a mixture of EC and DEC,
up to about 30% v/v of a mixture of EC and DEC,
up to about 50% v/v of a mixture of EC and DEC,
up to about 70% v/v of a mixture of EC and DEC, or
up to about 75% v/v of a mixture of EC and DEC,
   all w/w % being based on the total weight of the electrolyte and all v/v % being based on the total volume of the electrolyte.

In embodiments, the volume ratio of ethylene carbonate (EC) to diethyl carbonate (DEC) in the mixture of EC and DEC is from about 1:10 to about 1:1, preferably this volume ratio is about 3:7.

In preferred embodiments, the additives are ethylene carbonate and fluoroethylene carbonate only (preferably in the above-mentioned quantities).

In more preferred embodiments, the additive is fluoroethylene carbonate only (preferably in the above-mentioned quantity).

### Corrosion Inhibitors

In preferred embodiments, the electrolyte is free of corrosion inhibitors. Indeed, as noted above, one of the advantages of the carbonate compounds of formula (I) is that they are characterized by their low corrosiveness against aluminum current collectors, even at voltages higher than 4.2 V.

In alternative embodiments, the electrolyte further comprises one or more corrosion inhibitors. Non-limiting examples of corrosion inhibitors include LiPF6, lithium cyan fluorophosphates, lithium fluoro oxalatophosphates, LiDFOB, LiBF4, lithium fluoro cyanoborates, and LiBOB.

In embodiments, the corrosion inhibitors represent a total of at least about 1% and/or at most about 35% of the total weight of the electrolyte. In embodiments, the total amount of corrosion inhibitors represents at least about 1% w/w, at least about 2% w/w, at least about 5% w/w, or at least about 10% w/w, and/or at most about 35% w/w, at most about 25% w/w, at most about 20% w/w, at most about 15% w/w, at most about 10% w/w, or at most about 7% w/w of the total weight of the electrolyte.

### Minimum Concentration of Compound of Formula (I) in the Electrolyte

The skilled person would understand that the concentration of carbonate compound of formula (I) in the electrolyte will be influenced by various factors, such as the desired concentration of the conducting salt, and the quantity of the above additives and corrosion inhibitors.

Nevertheless, the electrolyte of the present invention should contain the carbonate compound of formula (I) in a concentration sufficient to achieve a desired anodic dissolution suppression.

In practice, the concentration of carbonate compound of formula (I) necessary to achieve suppression of anodic dissolution will vary depending on various factors, such as the intended operating voltage and presence of corrosion inhibitors. Generally, when corrosion inhibitors are present, a lower concentration of the carbonate compound of formula (I) will be needed to achieve a desired suppression of anodic dissolution.

In preferred embodiments, the carbonate compound of formula (I) represents at least about 25% v/v, preferably at least about 50% v/v, more preferably at least about 75% v/v, yet more preferably at least about 85% v/v, even more preferably at least about 90% v/v, and most preferably at least about 95%, of the total volume of the electrolyte.

In alterative embodiments in which the electrolyte comprises one or more corrosion inhibitors as described in the previous section, the carbonate compound of formula (I) can be present at lower concentrations. For example at a concentration of at least about 10% v/v, preferably at least about 15% v/v, more preferably at least about 20% v/v, yet more preferably at least about 25% v/v, and most preferably at least about 30%, based on the volume of the electrolyte.

In preferred embodiments, the electrolyte of the invention is free of other solvents. In other words, the only solvent in the electrolyte is the carbonate compound of formula (I).

### Remaining Components of the Batteries

As noted above the battery of the present invention is comprised of:

(a) a cathode comprising an aluminum current collector,
(b) an anode,
(c) a separator membrane separating the anode and the cathode, and
(d) a low-corrosiveness non-aqueous electrolyte in contact with the anode and the cathode.

As noted above, this battery, in preferred embodiments, is a lithium battery, a lithium-ion battery, a sodium battery, a sodium-ion battery, a potassium battery, a potassium-ion battery, a magnesium battery, a magnesium-ion battery, an aluminum battery, or an aluminum ion battery.

The choice of non-aqueous solvent, anode, cathode, and separator membrane will vary depending on the type of battery. If the battery is a lithium-ion battery, it would be more appropriate to choose, for example, an electrolyte comprising lithium salt, such as a lithium sulfonyl amide salt as a conducting salt. However, if the battery is a sodium-based battery, it would be more appropriate to choose, for example, an electrolyte comprising a sodium salt as a conducting salt.

The non-aqueous electrolyte is the electrolyte defined in the previous section.

The anode can be any anode typically used for a battery.

In preferred embodiments, the anode is one that is suitable for a lithium or a lithium ion battery. Such anodes are usually made of Li metal, carbonaceous materials (graphite, coke, and hard carbon), silicon and its alloys, tin and its alloys, antimony and its alloys, and/or lithium titanate (Li₄Ti₅O₁₂). These materials are usually mixed with a solvent, a polymer binder and electro-conductive additives—which include various forms of conductive carbon, such as carbon nanotubes and carbon black—and subsequently coated on a copper current collector in order to obtain the anode. In preferred embodiments, the anode is made of lithium metal or graphite.

As one advantage of using the electrolyte of the present invention is the prevention of anodic dissolution of aluminum current collectors, the cathode can be any cathode typically used for a battery that comprises an aluminum current collector.

In preferred embodiments, the cathode is one that is suitable for a lithium or a lithium ion battery. Such cathodes usually comprise lithium compounds. These lithium compounds are usually mixed with a solvent, polymer binder and electro-conductive additives—which include various forms of conductive carbon, such as carbon nanotubes and carbon black—and subsequently coated on an aluminum current collector in order to obtain the cathode. This aluminum current collector is susceptible to anodic dissolution at elevated potential, especially if the electrolyte contains non-passivating conducting salts. Such lithium compounds include lithiated oxides of transition metals like LCO (LiCoO₂), LNO (LiNiO₂), LMO (LiMn₂O₄), LiCoₓNi₁₋ₓO₂ wherein the x is from 0.1 to 0.9, LMN (LiMn_{3/2}Ni_{1/2}O₄), LMC (LiMnCoO₂), LiCuₓMn₂₋ₓO₄, NMC (LiNiₓMn_{y}Co_{z}O₂), NCA (LiNiₓCo_{y}Al_{z}O₂), lithium compounds with transition metals and complex anions, LFP (LiFePO₄), LNP (LiNiPO₄), LMP (LiMnPO₄), LCP (LiCoPO₄), Li₂FCoPO₄; LiCo_{q}FeₓNi_{y}Mn_{z}PO₄, and Li₂MnSiO₄.

In preferred embodiments, the cathode of the present invention is an LMN cathode or an LCO cathode.

In embodiments, the cathode comprises only the current collector.

In embodiments, the cathode is made by coating the current collector with the above described lithium compounds, preferably LMN or LCO. In preferred embodiments, the current collector is an aluminum current collector.

In order to prevent physical contact between electrodes, a separator membrane is usually placed between them. The separator membrane can be any separator membrane typically used for a battery.

In preferred embodiments, the separator membrane is one that is suitable for a lithium or a lithium ion battery. Mother function of such a separator membrane is to prevent lithium dendrite from causing a short-circuit between electrodes. Such separator membranes typically include (i) a polyolefin based porous polymer membrane, preferably made of polyethylene “PE”, polypropylene “PP”, or a combination of PE and PP, such as a trilayer PP/PE/PP membrane; (ii) heat-activatable microporous membranes; (iii) porous materials made of fabric including glass, ceramic or synthetic fabric (woven or non-woven fabric); (iv) porous membranes made of polymer materials such as polyvinyl alcohol), polyvinyl acetate), cellulose, and polyamide; (v) porous polymeric membranes provided with an additional ceramic layer in order to improve the performance at high potentials; and (vi) polymer electrolyte membranes. However, as mentioned, the separator membrane can also be any separator membrane typically used for a battery, preferably for a lithium or a lithium ion battery; for example Celgard 3501™ or Celgard Q20S1HX™.

Depending on the type of battery, a different cathode, anode, and separator membrane may be provided or prepared. Much like the electrolyte, the cathode, anode, and separator membrane can be prepared using any known technique in the art, and the battery can be prepared using any known technique in the art.

As noted above, the batteries of the present invention have a wide variety of applications that would be readily understood by the person of skill in the art. Such applications include electric vehicles, power tools, grid energy storage, medical devices and equipment, toys, hybrid electric vehicles, cell phones, laptops, and various military and aerospace applications.

### Method of Producing the Carbonate Solvents, the Non-Aqueous Electrolytes, and the Batteries

In another aspect of the invention, a method for producing the above carbonate solvents and batteries is provided.

Each of the carbonate solvent, the electrolyte, and the battery of the present invention can be prepared using any known technique in the art.

For example, the carbonate solvents of the invention can be synthesised according to the following formula:

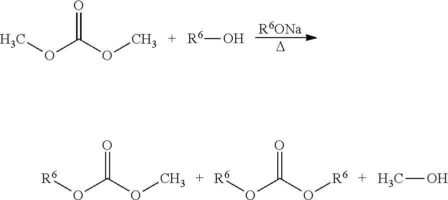

In the above formula, R⁶ represents both R¹ and R², defined above. Syntheses of alkyl carbonates are very well-developed processes. While the most convenient methods are discussed below, the skilled person would understand that other synthesis methods can be used.

Preparation of the carbonate solvents of the present invention in smaller scale is most conveniently accomplished by a base catalyzed transesterification of readily available dimethyl carbonate, diethyl carbonate, ethylene carbonate, or propylene with aliphatic alcohols in the presence of a suitable catalyst. The transesterification of carbonate esters obeys the same rules as transesterification of other esters, which is a typical equilibrium reaction, and can be easily controlled by the use of Le Chateliers' principle. The ratio between the alcohol and the carbonate ester determines the ratio of the products in a fully equilibrated reaction mixture. If a full substitution is desired, the excess of alcohol should be used. If the desired product is the mixed carbonate, the molar ratio should be close to 1, or a slight excess of starting carbonate should be used. During the reaction, it is desirable that the reaction products are steadily removed from the reaction mixture; this allows the reaction to proceed faster to completion. The separation is most conveniently done by fractional distillation of a lower alcohol. For this reason, the use of low carbonates is preferred over higher carbonates because the formed alcohol has a lower boiling point; however, attention must be paid to the formation of azeotropic mixtures which may complicate the separation.

The catalysts used for this transformation can be chosen from acids and from bases, but bases like alkali and earth alkali carbonates, oxides, hydroxides and alkoxides are preferred as they can be separated easily from the volatile products.

In a suitable reaction vessel equipped for a fractioning distillation, there is placed the appropriate amount of desired aliphatic alcohol and a certain amount of metallic sodium is added. The amount of sodium should be chosen so that it will react with the water present in the reactants and consume it all. In this way, a water free solvent can be isolated. The process of sodium dissolution can be accelerated by heating and stirring, which is necessary with all higher alcohols. A protective atmosphere of nitrogen or argon should be used to exclude the uptake of carbon dioxide and water from the atmosphere. When sodium is dissolved, the starting carbonate ester is added and the mixture is refluxed at such a temperature that the alcohol which is formed during the reaction distills from the reaction mixture, while all reactants remain in the reactor. After the reaction is finished, the components of the reaction mixture are separated by fractional distillation, under vacuum for higher alkyl carbonates. In this manner the solvents can be isolated in high purity if no azeotropes are formed.

However, the industrial preparation of symmetric carbonates can be performed by phosgenation of the corresponding alcohols.

When small amounts of mixed carbonate are desired, the most suitable method seems to be the reaction of an aliphatic alcohol with an aliphatic chloroformate in an aprotic solvent in the presence of a suitable base which binds the formed HCl. Even though this method is well established, it may sometimes give erroneous results.

### Definitions

The use of the terms “a” and “an” and “the” and similar referents in the context of describing the invention (especially in the context of the following claims) are to be construed to cover both the singular and the plural, unless otherwise indicated herein or dearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Similarly, herein a general chemical structure with various substituents and various radicals enumerated for these substituents is intended to serve as a shorthand method of referring individually to each and every molecule obtained by the combination of any of the radicals for any of the substituents. Each individual molecule is incorporated into the specification as if it were individually recited herein. Further, all subsets of molecules within the general chemical structures are also incorporated into the specification as if they were individually recited herein.

All methods described herein can be performed in any suitable order unless otherwise indicated herein or otherwise dearly contradicted by context.

The use of any and all examples, or exemplary language (e.g., “such as”) provided herein, is intended merely to better illuminate the invention and does not pose a limitation on the scope of the invention unless otherwise claimed.

No language in the specification should be construed as indicating any non-claimed element as essential to the practice of the invention.

Herein, the term “about” has its ordinary mewing. In embodiments, it may mean plus or minus 10% or plus or minus 5% of the numerical value qualified.

Unless otherwise defined, all technical and scientific terms used herein have the same meaning as commonly understood by one of ordinary skill in the at to which this invention belongs.

For certainty, it should be noted that
alkyloyl is alkyl-C(═O)—,
aryloyl is aryl-C(═O)—,
alkyloxycarbonyl is alkyl-O—C(═O)—, and
ayloxycarbonyl is aryl-O—C(═O)—.

Herein, the terms “alkyl” has its ordinary meaning in the art. It is to be noted that, unless otherwise specified, the hydrocarbon chain of the alkyl groups can be linear or branched.

Herein, the terms “aryl” has its ordinary meaning in the art. It is to be noted that, unless otherwise specified, the aryl groups can contain between 5 and 30 atoms, including carbon and heteroatoms, preferably without heteroatoms, more specifically between 5 and 10 atoms, or contain 5 or 6 atoms.

For clarity, the following abbreviations are used: EC—ethylene carbonate, PC—propylene carbonate, DEC—diethyl carbonate, EMC—ethyl methyl carbonate, DMC—methyl carbonate, FEC—fluoroethylene carbonate, VC—vinylene carbonate, LCO—LiCoO₂-lithium cobaltate, LMN—LiMn_{3/2}Ni_{1/2}O₄,

Other objects, advantages and features of the present invention will become more apparent upon reading of the following non-restrictive description of specific embodiments thereof, given by way of example only with reference to the accompanying drawings.

### DESCRIPTION OF ILLUSTRATIVE EMBODIMENT

The present invention is illustrated in further details by the following non-limiting examples.

A brief summary of the nature of each Example is as follows:

Examples 1-4 involve the preparation of various carbonate solvents of the present invention.

Examples 5-7 are comparative examples wherein anodic dissolution is measured in button cells comprising conventional electrolytes.

Examples 8-10 measure anodic dissolution in button cells comprising electrolytes of the present invention.

Examples 11-54 involve measuring the starting potentials of anodic dissolution of various electrolytes of the present invention.

Examples 55 and 58 are comparative examples where charging and discharging of button cells comprising conventional electrolytes was measured.

Examples 56, 57, and 59 involve measuring the charging and discharging of button cells comprising electrolytes of the present invention.

Example 60 involves measuring the temperature range of an electrolyte of the present invention and a conventional electrolyte by performing a digital scanning calorimetry (DSC) experiment.

Example 61 involves measuring the discharge capacity of three cells versus cycle number; two of the cells comprise electrolytes of the present invention, while one comprises a conventional electrolyte.

### Preparation of Carbonate Solvents of the Invention

### Example 1: Preparation of Diisobutyl Carbonate (Solvent No. 10) by Transesterification

In a 250 ml round bottom flask equipped with a Vigreux column is placed 128 g (1.73 mol) of isobutanol, in which 0.3 g sodium was dissolved at the baling pant and 59 g (0.66 mol) of dimethyl carbonate was added. The mixture was refluxed overnight with the separation of methanol formed. After the separation of the methanol ceased, the remainder was subjected to a fractional distillation, giving 120 g of diisobutyl carbonate as a colourless liquid. Unreacted isobutanol, dimethyl carbonate and isobutyl methyl carbonate were also detected in the preceding fraction. The structure of the products was confirmed by NMR (nuclear magnetic resonance spectroscopy), IR (infra-red spectroscopy) and GC/MS (gas chromatography with mass selective detector) analyses.

### Example 2: Preparation of di(2-pentyl) Carbonate (Solvent No. 17) and methyl 2-pentyl Carbonate (Solvent No. 16) by Transesterification

In a 250 ml round bottom flask equipped with a Vigreux column is placed 116 g (1316 mmol) of 2-pentanol, in which 1 g sodium was dissolved at 100° C. and 98 g (1088 mmol) of dimethyl carbonate was added. The mixture was refluxed over 48 h with the separation of methanol formed. After the separation of the methanol ceased, the remainder was subjected to a vacuum fractional distillation, giving a smaller fraction of 40 g containing pure methyl 2-pentyl carbonate and a main fraction of 70 g of di(2-pentyl) carbonate as a colourless liquid. Unreacted dimethyl carbonate, 2-pentanol and methanol were also detected in the preceding fractions. The structure of the products was confirmed by NMR, IR and GC/MS analyses.

### Example 3: Preparation of 2-cyanoethyl Butyl Carbonate (Solvent No. 23) by Tendon of Butyl Chloroformate and 3-Hydroxypropinonitril

In a 250 ml round bottom flask is placed 7.1 g (100 mmol) of 3-hydroxypropinonitril, 11 g of a freshly distilled triethyl amine, and 150 ml dry dichloromethane. The mixture was cooled under nitrogen in an ice water bath and a solution of 13.66 g (100 mmol) of butyl chloroformate in 30 ml of dichloromethane was added dropwise. After the addition, the mixture was stirred at room temperature for 2 h, after which water and sulfuric acid were added and the mixture was separated by means of a separation funnel. The organic phase was washed 4 times with water, and then dried with anhydrous magnesium sulfate, filtered, and evaporated. Distillation of the remaining clear oil gave pure product (13.1 g). Structure of the products was confirmed by NMR, IR and GC/MS analyses.

### Example 4: Preparation of Other Carbonate Solvents of the Present Invention

Other dialkylcarbonates were prepared similarly to the procedures in examples 1-3 and all are listed in the following table (Table 1) together with their ¹H and ¹³C NMR spectroscopy data.

TABLE 1

| | | 1H NMR | 13C NMR |
|---|---|---|---|
| Solvent No | Name | (400 MHz, CHLOROFORM-d) δ= | (101 MHz, CHLOROFORM-d) δ= |
| 1 | didodecyl carbonate | 4.10 (t, J = 6.8 Hz, 2H), 1.65 (br quin, | 155.39, 67.92, 31.88, 29.61, |
| | | J = 6.8 Hz, 2H), 1.40-1.22 (m, 18H), | 29.59, 29.53, 29.46, 29.32, 29.21, |
| | | 0.87 (br t, J = 6.8 Hz, 3H) | 28.67, 25.68, 22.64, 14.03 |
| 2 | dibutyl carbonate | (400 MHz, DICHLOROMETHANE-d₂) δ = | (101 MHz, |
| | | 4.07 (t, J = 6.7 Hz, 1H), 1.60 (br quin, | DICHLOROMETHANE-d₂) δ = |
| | | J = 7.3 Hz, 1H), 1.37 (br sxt, J = 7.5 Hz, | 156.02, 68.06, 31.45, 19.62, |
| | | 1H), 0.91 (t, J = 7.4 Hz, 1H) | 14.07 |
| 3 | dipropyl carbonate | ¹H NMR (400 MHz, | (101 MHz, |
| | | DICHLOROMETHANE-d₂) δ = 4.04 (t, | DICHLOROMETHANE-d₂) δ = |
| | | J = 6.7 Hz, 2H), 1.66 (sxt, J = 7.1 Hz, 2H), | 155.98, 69.83, 22.66, 10.53 |
| | | 0.93 (t, J = 7.4 Hz, 3H) | |
| 4 | methyl propyl carbonate | 3.90 (t, J = 6.7 Hz, 2H), 3.57 (s, 3H), 1.50 | 155.42, 68.95, 53.88, 21.56, 9.54 |
| | | (sxt, J = 7.1 Hz, 2H), 0.77 (t, J = 7.5 Hz, | |
| | | 3H) | |
| 5 | diisopropyl carbonate | (400 MHz, DICHLOROMETHANE-d₂) δ = | (101 MHz, |
| | | 4.79 (spt, J = 6.3 Hz, 1H), 1.23 (d, | DICHLOROMETHANE-d₂) δ = |
| | | J = 6.3 Hz, 6H) | 154.79, 71.68, 22.18 |
| 6 | isopropyl methyl | 4.70 (spt, J = 6.4 Hz, 1H), 3.59 (s, 3H), | 154.92, 71.35, 53.84, 21.29 |
| | carbonate | 1.13 (d, J = 6.4 Hz, 6H) | |
| 7 | ethyl dodecyl carbonate | 4.16 (q, J = 7.3 Hz, 2H), 4.09 (t, J = 6.7 Hz, | 155.17, 67.81, 63.56, 31.81, |
| | | 2H), 1.63 (quin, J = 7.1 Hz, 2H), 1.28 (t, | 29.53, 29.52, 29.45, 29.40, 29.24, |
| | | J = 7.2 Hz, 3H), 1.39-1.20 (m, 18H), | 29.13, 28.59, 25.61, 22.57, 14.13, |
| | | 0.85 (t, J = 6.9 Hz, 3H) | 13.95 |
| 8 | ethyl propyl carbonate | 4.03 (q, J = 7.1 Hz, 2H), 3.94 (t, J = 6.7 Hz, | 154.93, 68.91, 63.26, 21.70, |
| | | 2H), 1.55 (sxt, J = 7.1 Hz, 2H), 1.15 (t, | 13.84, 9.74 |
| | | J = 7.2 Hz, 3H), 0.82 (t, J = 7.5 Hz, 3H) | |
| 9 | ethyl isopropyl carbonate | 4.68 (spt, J = 6.4 Hz, 1H), 4.00 (q, J = 7.1 | 154.26, 71.00, 63.18, 21.29, |
| | | Hz, 2H), 1.13-1.09 (m, 9H) | 13.80 |
| 10 | diisobutyl carbonate | 3.80 (d, J = 6.8 Hz, 2H), 1.87 (nonuplet, | 155.29, 73.57, 27.58, 18.63 |
| | | J = 6.7, 1H), 0.85 (d, J = 7.1 Hz, 6H) | |
| 11 | isobutyl methyl | 3.78 (d, J = 6.6 Hz, 2H), 3.63 (s, 3H), | 155.59, 73.65, 54.11, 27.47, |
| | carbonate | 1.83 (nonuplet, J = 6.8 Hz, 1H), 0.81 (d, | 18.45 |
| | | J = 6.8 Hz, 6H) | |
| 12 | dipentyl carbonate | 4.03 (t, J = 6.7 Hz, 2H), 1.59 (quin, J = 6.9 | 155.20, 67.64, 28.19, 27.65, |
| | | Hz, 2H), 1.34-1.19 (m, 4H), 0.82 (br t, | 22.08, 13.62 |
| | | J = 6.8 Hz, 3H) | |
| 13 | methyl pentyl carbonate | 4.04 (t, J = 6.7 Hz, 2H), 3.68 (s, 3H), 1.58 | 155.67, 67.89, 54.24, 28.15, |
| | | (quin, J = 7.0 Hz, 2H), 1.35-1.13 (m, | 27.60, 22.05, 13.61 |
| | | 4H), 0.82 (t, J = 7.0 Hz, 3H) | |
| 14 | di(2-ethylhexyl) | 4.11-3.93 (m, 2H), 1.68-1.53 (m, 1H), | 155.68, 70.25, 38.81, 30.07, |
| | carbonate | 1.48-1.13 (m, 8H), 0.88 (t, J = 7.5 Hz, | 28.82, 23.44, 22.88, 13.92, 10.80 |
| | | 6H) | |
| 15 | 2-ethylhexyl methyl | 4.04-3.89 (m, 2H), 3.67 (s, 3H), 1.59- | 155.76, 70.10, 54.19, 38.68, |
| | carbonate | 1.41 (m, 1H), 1.36-1.06 (m, 8H), 0.81 | 29.94, 28.67, 23.29, 22.70, 13.69, |
| | | (br t, J = 7.5 Hz, 6H) | 10.60 |
| 16 | methyl 2-pentyl | 4.67 (sxt, J = 6.2 Hz, 1H), 3.66 (s, 3H), | 155.27, 74.85, 54.06, 37.76, |
| | carbonate | 1.61-1.46 (m, 1H), 1.45-1.19 (m, 3H), | 19.59, 18.28, 13.54 |
| | | 1.17 (d, J = 6.1 Hz, 3H), 0.82 (t, J = 7.2 Hz, | |
| | | 3H) | |
| 17 | di(2-pentyl) carbonate | 4.60 (sxt, J = 6.2 Hz, 1H), 1.55-1.41 (m, | 154.29, 74.07, 37.77, 19.54 (d, |
| | | 1H), 1.39-1.14 (m, 3H), 1.10 (d, J = 6.1 | J = 2.2 Hz), 18.27 (br d, J = 2.2 Hz), |
| | | Hz, 3H), 0.77 (t, J = 7.2 Hz, 3H) | 13.46 |
| 18 | 2-butyl methyl carbonate | 4.52 (sxt, J = 6.2 Hz, 1H), 3.59 (s, 3H), | 155.14, 75.98, 53.84, 28.37, |
| | | 1.71-1.29 (m, 2H), 1.09 (d, J = 6.4 Hz, | 18.87, 9.07. |
| | | 3H), 0.76 (t, J = 7.5 Hz, 3H) | |
| 19 | di(2-butyl) carbonate | 4.58 (sxt, J = 6.3 Hz, 1H), 1.63-1.39 (m, | 154.45, 75.68 (br d, J = 1.5 Hz), |
| | | 2H), 1.16 (d, J = 6.4 Hz, 3H), 0.83 (t, | 28.59, 19.14 (br d, J = 3.7 Hz), |
| | | J = 7.5 Hz, 3H) | 9.35 (br d, J = 2.9 Hz) |
| 20 | 2-ethylbutyl methyl | 4.01 (d, J = 5.6 Hz, 2H), 3.72 (s, 3H), | 155.88, 69.93, 54.38, 40.27, |
| | carbonate | 1.49 (spt, J = 6.1 Hz, 1H), 1.33 (quin, | 22.90, 10.75 |
| | | J = 7.2 Hz, 4H), 0.85 (t, J = 7.5 Hz, 6H) | |
| 21 | di(2-ethylbutyl) | 3.99 (d, J = 6.1 Hz, 2H), 1.50 (spt, J = 6.1 | 155.58, 69.73, 40.27, 22.88, |
| | carbonate | Hz, 1H), 1.32 (nonuplet, J = 7.3, 14.6 Hz, | 10.71 |
| | | 4H), 0.84 (t, J = 7.6 Hz, 6H) | |
| 22 | isobutyl isopropyl | 4.83 (spt, J = 5.9 Hz, 1H), 3.86 (d, J = 6.4 | 154.75, 73.54, 71.43, 27.67, |
| | carbonate | Hz, 2H), 1.93 (nonuplet, J = 6.5 Hz, 1H), | 21.63, 18.79 |
| | | 1.25 (d, J = 6.1 Hz, 6H), 0.91 (d, J = 6.8 | |
| | | Hz, 6H) | |
| 23 | 2-cyanoethyl butyl | 4.26 (t, J = 6.2 Hz, 1H), 4.10 (t, J = 6.7 Hz, | 154.28, 116.42, 68.18, 61.51, |
| | carbonate | 1H), 2.70 (t, J = 6.2 Hz, 1H), 1.60 (quin, | 30.24, 18.55, 17.74, 13.28 |
| | | J = 7.1 Hz, 1H), 1.34 (sxt, J = 7.4 Hz, 1H), | |
| | | 0.88 (t, J = 7.3 Hz, 2H) | |
| 24 | 2-methoxyethyl isobutyl | 4.23-4.19 (m, 2H), 3.85 (d, J = 6.6 Hz, | 155.12, 73.86, 70.00, 66.43, |
| | carbonate | 2H), 3.57-3.53 (m, 2H), 3.32 (s, 3H), | 58.69, 27.54, 18.65 |
| | | 1.90 (nonuplet, J = 6.6 Hz, 1H), 0.88 (d, | |
| | | J = 6.6 Hz, 6H) | |
| 25 | (2-trimethylsilyloxy)ethyl | 4.12 (t, J = 4.8 Hz, 2H), 4.07 (t, J = 6.4 Hz, | 155.15, 68.48, 67.59, 60.32, |
| | butyl carbonate | 2H), 3.73 (t, J = 4.9 Hz, 2H), 1.58 (quin, | 30.50, 18.71, 13.41, −0.78 |
| | | J = 7.1 Hz, 2H), 1.34 (sxt, J = 7.4 Hz, 2H), | ²⁹Si NMR (79 MHz, |
| | | 0.87 (t, J = 7.3 Hz, 3H), 0.06 (s, 9H) | CHLOROFORM-d) δ = 19.31. |
| 26 | di(2-methoxyethyl) | 4.18-4.05 (m, 2H), 3.52-3.40 (m, 2H), | 154.70, 69.69, 66.41, 58.37 |
| | carbonate | 3.22 (s, 3H) | |
| 27 | 2-isopropoxyethyl | 4.15-4.02 (m, 2H), 3.61 (s, 3H), 3.50- | 155.37, 71.51, 66.96, 65.28, |
| | methyl | 3.46 (m, 2H), 3.45 (spt, J = 6.1 Hz, 1H), | 54.16, 21.49 |
| | carbonate | 0.99 (d, J = 6.1 Hz, 6H) | |
| 28 | di(2-isopropoxyethyl) | 4.32-4.01 (m, 2H), 3.56-3.50 (m, 2H), | 154.93, 71.65, 67.01, 65.35, |
| | carbonate | 3.49 (spt, J = 6.1 Hz, 1H), 1.04 (d, J = 6.2 | 21.65 |
| | | Hz, 6H) | |
| 29 | di(2-(2- | 4.06 (t, J = 4.5 Hz, 2H), 3.50 (t, J = 4.5 Hz, | 154.44, 71.23, 69.86, 68.24, |
| | methoxyethoxy)ethyl) | 2H), 3.46-3.39 (m, 2H), 3.36-3.27 (m, | 66.33, 58.30 |
| | carbonate | 2H), 3.15 (s, 3H) | |

### Measurement of Anodic Dissolution of Aluminum Current Collector

In the following examples, anodic dissolution of an aluminum current collector was measured. Detection of anodic dissolution of an aluminum current collector can be realised by many electrochemical methods. One indicator of anodic dissolution is the current which appears between the reference electrode and the bae aluminum electrodes at a certain potential. Anodic dissolution is strongly dependent on the applied potential, so the variation of the potential during anodic dissolution probing is essential.

In light of the above, in the following Examples, anodic dissolution of an aluminum current collector was measured using chronoamperometry, CA. Chronoamperometry involves measuring the current at a given potential and is usually performed over a longer period of time; accordingly, even the slowest processes can be detected in that manner. For the following examples, chronoamperometry was used for 1 h at potentials between 4-5.5 V vs Li metal by 0.1 V steps (1 hour of CA at 4.0, 4.1, 4.2, etc., until 5.5 V). This enables relatively fast screening of the electrolytes.

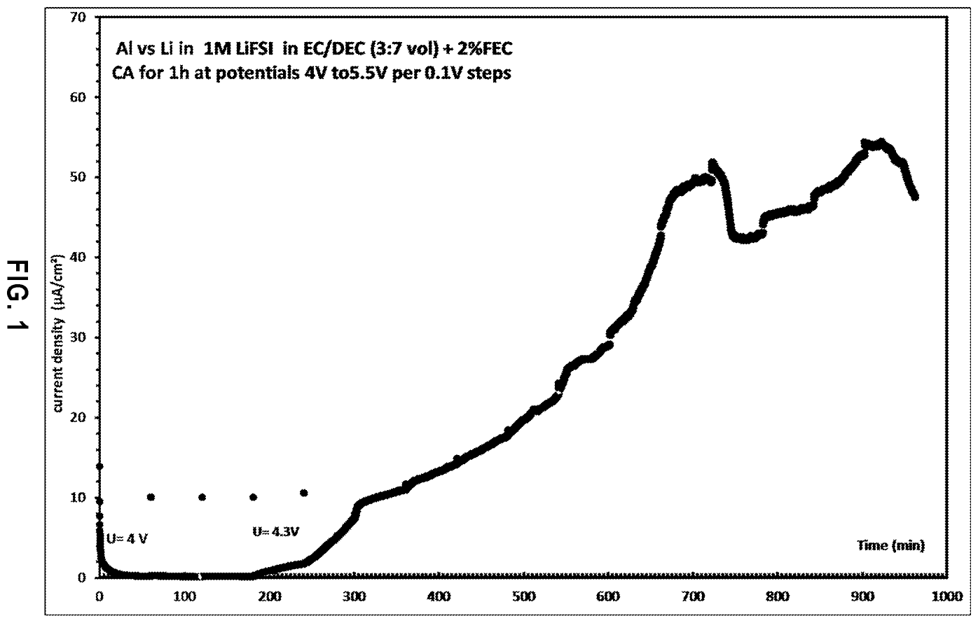

The chronoamperometry results are shown in FIGS. 1 to 6.

In general, it was found that in electrolytes obtained by dissolution of LiFSI, LiFTFSI and LiTFSI in mixtures of dimethyl carbonate (DMC), diethyl carbonate (DEC), ethyl methyl carbonate (EMC), ethylene carbonate (EC) and propylene carbonate (PC), significant anodic dissolution appeared between 4.1 and 4.3 V vs Li metal, detected as a high current/current density between electrodes (FIGS. 1-3).

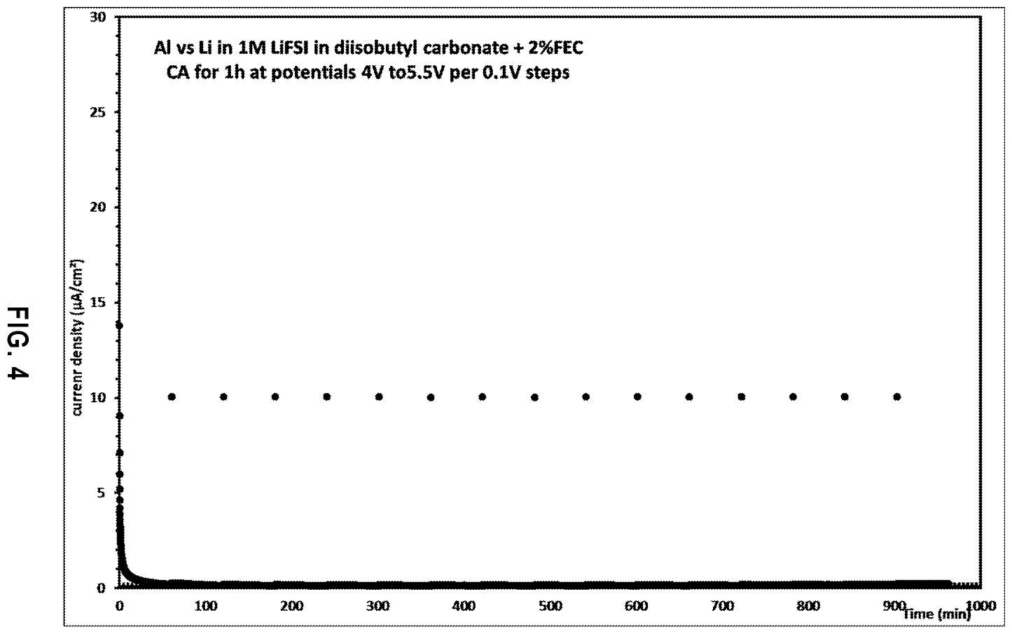

However, it was found that LiFSI, LiFTFSI and LiTFSI, when dissolved in higher, preferably branched, dialkyl carbonate, where the total number of carbon atoms was equal to or greater than 4, did not cause anodic dissolution of the aluminum current collector, in some cases even at potentials over 5 V vs Li metal (FIGS. 4-6).

### Example 5 (Comparative): Anodic Dissolution of Aluminum Current Collector in LiFSI-EC-DEC Electrolyte

1M solution of LiFSI (Nippon Shokubai) in a conventional industrial solvent mixture of ethylene carbonate and diethyl carbonate (EC/DEC), in volume ratio of 3:7, was prepared and 2 wt % of fluoroethylene carbonate was added.

A button cell was assembled using a disc of 16 mm diameter, with a 15 μm thickness of non-coated aluminum current collector, provided by UACJ as a cathode. Celgard 3501 was used as a separator membrane and the aforementioned LiFSI-EC-DEC electrolyte was also used. A 16 mm, 200 μm thick disc of lithium metal, provided by China Energy Lithium Co., LTD., was used as an anode. The cell was used for probing the anodic dissolution of the cathode during chronoamperometry for 1 h at potentials between 4 and 5.5 V vs Li metal at 0.1 V steps (1 hour of chronoamperometry at 4.0, 4.1, 4.2 . . . 5.5 V). The results of this experiment can be seen in FIG. 1. Already at 4.3 V a significant appearance of current is observed, which indicates anodic dissolution. Accordingly, this electrolyte cannot be used for batteries where the potential of the cathode surpasses 4.3 V.

### Example 6 (Comparative): Mock Dissolution of Aluminum Current Collector in LiFTFSI-EC-DEC Electrolyte

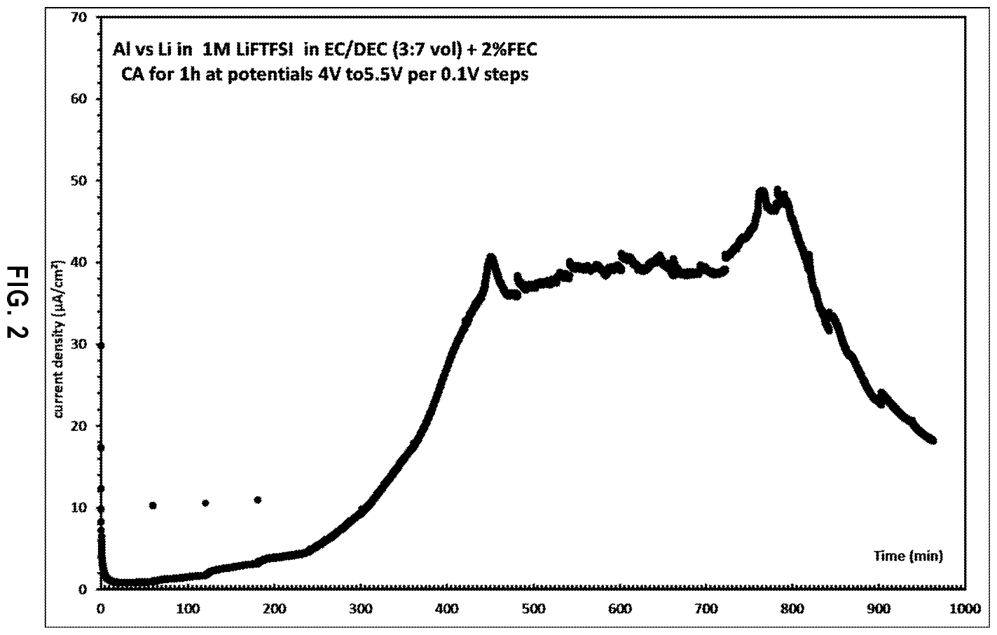

A button cell was assembled and tested according to example 5 but using a 1M solution of LiFTFSI in a conventional industrial solvent mixture of ethylene carbonate/diethyl carbonate, EC/DEC, in a volume ratio of 3:7, and 2 wt % of fluoroethylene carbonate, as an electrolyte. The results of this experiment can be seen in FIG. 2. Already at 4.1 V a significant appearance of current is observed, which indicates anodic dissolution. Accordingly, this electrolyte cannot be used for batteries where the potential of the cathode surpasses 4.2 V.

### Example 7 (Comparative): Anodic Dissolution of Aluminum Current Collector in LiTFSI-EC-DEC Electrolyte

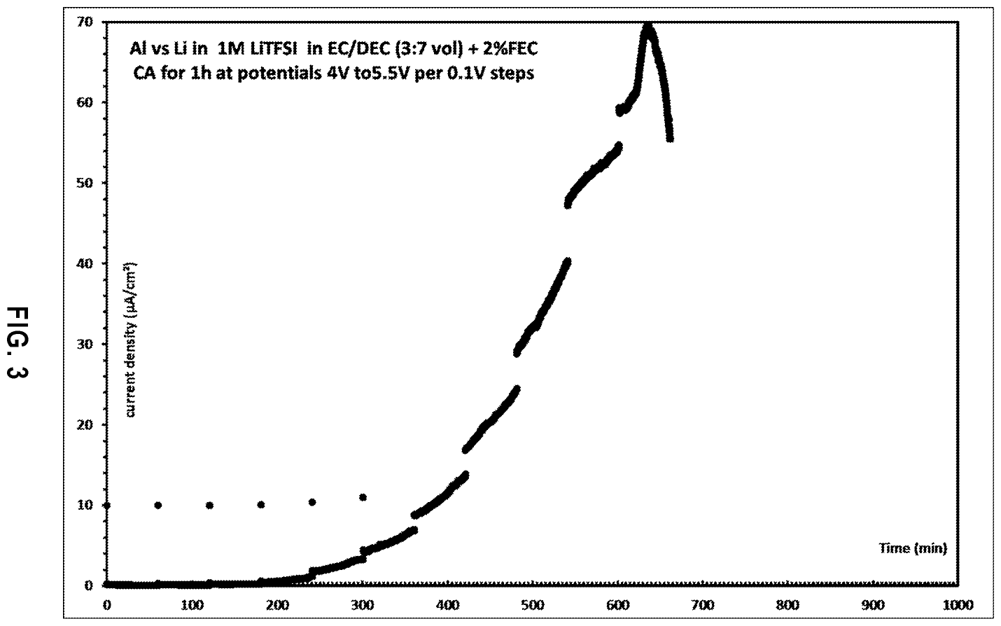

A button cell was assembled and tested according to example 5 but using a 1M solution of LiTFSI (available from 3M™) in a conventional industrial solvent mixture of ethylene carbonate and diethyl carbonate, EC/DEC, in a volume ration of 3:7, and 2 wt % of fluoroethylene carbonate, as an electrolyte. The results of this experiment can be seen on FIG. 3. Already at 4.1 V, a significant appearance of current is observed, which indicates anodic dissolution. Accordingly, this electrolyte cannot be used for batteries where the potential of the cathode surpasses 4.2 V.

### Example 8: Suppression of Anodic Dissolution of Aluminum Current Collector in LiFSI-Diisobutyl Carbonate Electrolyte

1M solution of LiFSI (Nippon Shokubai) in diisobutyl carbonate (Solvent no. 10 prepared according to Example 1) was prepared, to which 2 wt % of fluoroethylene carbonate was added.

A button cell was assembled and tested according to Example 5 but using the preceding LiFSI-diisobutyl carbonate electrolyte. The results of this experiment can be seen in FIG. 4. On all potentials tested (4-5.5V), the current density stays well below 1 μA/cm², meaning this electrolyte can be used inter alia with cathodes with a cut off potential of at least at 5.5 V.

### Example 9: Suppression of Anodic Dissolution of Aluminum Current Collector in LiFTFSI-Diisobutyl Carbonate Electrolyte

1M solution of LiFTFSI in diisobutyl carbonate (Solvent no. 10) was prepared according to example 1, to which 2 wt % of fluoroethylene carbonate was added.

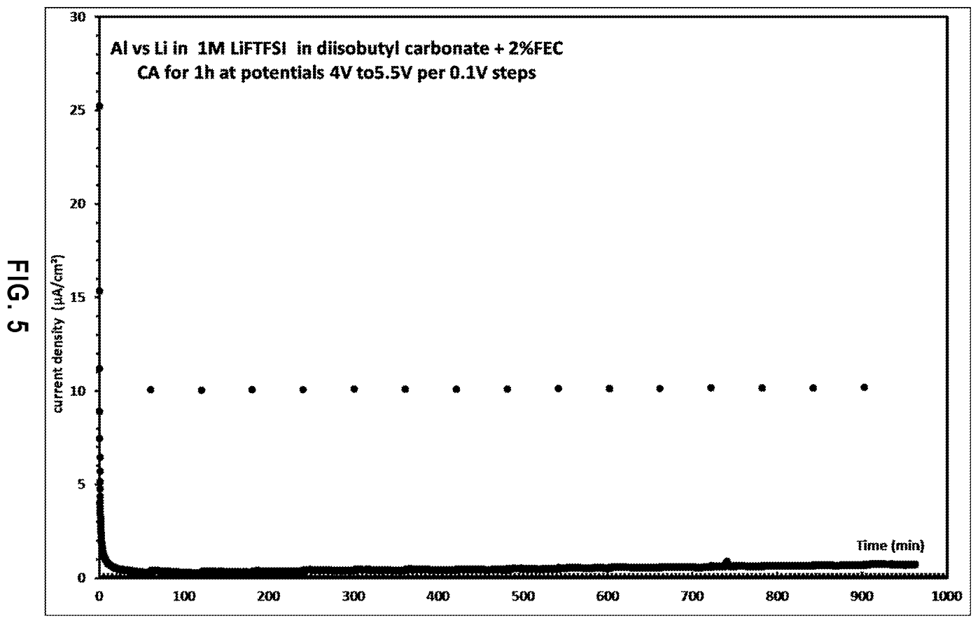

A button cell was assembled and tested according to example 5 but using the preceding LiFTFSI-diisobutyl carbonate electrolyte. The results of this experiment can be seen in FIG. 5. On all potentials tested (4-5.5V), the current density stays well below 1 μA/cm², meaning this electrolyte can be used inter alia in battery systems where the voltage surpasses 5.5 V. As mentioned, electrolytes prepared from conventional solvents containing FSI typically become unsafe when the operating voltage surpasses 4.3 V (see Examples 5 to 7).

### Example 10: Suppression of Anodic Dissolution of Aluminum Current Collector in LiTFSI-Diisobutyl Carbonate Electrolyte

1M solution of LiTFSI in diisobutyl carbonate (Solvent no. 10) was prepared according to example 1, to which 2 wt % of fluoroethylene carbonate was added.

A button cell was assembled and tested according to example 5 but using the preceding LiTFSI-diisobutyl carbonate electrolyte. The results of this experiment can be seen in FIG. 6. On all potentials tested (4-5.5V), the current density stays below 1 μA/cm², mewing this electrolyte can be used inter alia in battery systems where the voltage surpasses 5.5 V.

### Examples 11-54: Starting Potentials of Anodic Dissolution of Various Electrolytes

Button cells were assembled and tested according to example 5 but using each of the electrolytes listed in the following table (Table 2) with the previous results. The results of this experiment are presented as the potential where significant anodic dissolution of aluminum occurs and represents a safe use limit for said electrolyte. Several examples have been made in order to illustrate the mixing possibilities of different solvents in order to get an electrolyte with enhanced conductivity, while maintaining the effect of suppressing anodic dissolution.

In the table below, “EC/DEC (3:7 vol)” denotes an EC/DEC mixture in a 3:7 volume ratio.

TABLE 2

| | | | | Starting potential |
|---|---|---|---|---|
| | Solvent No. | | Conc. | of anodic |
| Example | (from table 1) | Additives | Salt | dissolution [V] |
| 5 (comp) | none | EC/DEC (3:7 vol) + | 1M LiFSI | 4.3 |
| | | 2 wt % FEC | | |
| 6 (comp) | none | EC/DEC (3:7 vol) + | 1M LiFTFSI | 4.2 |
| | | 2 wt % FEC | | |
| 7 (comp) | none | EC/DEC (3:7 vol) + | 1M LiTFSI | 4.2 |
| | | 2 wt % FEC | | |
| 8 | 10 | 2 wt % FEC | 1M LiFSI | >5.5 |
| 9 | 10 | 2 wt % FEC | 1M LiFTFSI | >5.5 |
| 10 | 10 | 2 wt % FEC | 1M LiTFSI | >5.5 |
| 11 | 1 | 2 wt % FEC | 1M LiFSI | >5.5 |
| 12 | 2 | 2 wt % FEC | 1M LiFSI | 5 |
| 13 | 3 | 2 wt % FEC | 1M LiFSI | 4.7 |
| 14 | 4 | 2 wt % FEC | 1M LiFSI | 4.5 |
| 15 | 5 | 2 wt % FEC | 1M LiFSI | 4.8 |
| 16 | 6 | 2 wt % FEC | 1M LiFSI | 4.6 |
| 17 | 7 | 2 wt % FEC | 1M LiFSI | >5.5 |
| 18 | 8 | 2 wt % FEC | 1M LiFSI | 4.5 |
| 19 | 9 | 2 wt % FEC | 1M LiFSI | 4.5 |
| 20 | 11 | 2 wt % FEC | 1M LiFSI | 4.6 |
| 21 | 12 | 2 wt % FEC | 1M LiFSI | 4.9 |
| 22 | 13 | 2 wt % FEC | 1M LiFSI | 4.5 |
| 23 | 14 | 2 wt % FEC | 1M LiFSI | >5.5 |
| 24 | 15 | 2 wt % FEC | 1M LiFSI | 5.0 |
| 25 | 16 | 2 wt % FEC | 1M LiFSI | 4.7 |
| 26 | 17 | 2 wt % FEC | 1M LiFSI | 5.5 |
| 27 | 18 | 2 wt % FEC | 1M LiFSI | 4.7 |
| 28 | 19 | 2 wt % FEC | 1M LiFSI | 5.3 |
| 29 | 20 | 2 wt % FEC | 1M LiFSI | 5 |
| 30 | 21 | 2 wt % FEC | 1M LiFSI | 5.1 |
| 31 | 22 | 2 wt % FEC | 1M LiFSI | 4.5 |
| 32 | 10 | 5 v/v % EC + | 1M LiFSI | >5.5 |
| | | 2 wt % FEC | | |
| 33 | 10 | 10 v/v % EC + | 1M LiFSI | 5.2 |
| | | 2 wt % FEC | | |
| 34 | 10 | 15 v/v % EC + | 1M LiFSI | 4.4 |
| | | 2 wt % FEC | | |
| 35 | 10 | 20 v/v % EC + | 1M LiFSI | 4.2 |
| | | 2 wt % FEC | | |
| 36 | 10 | 30 v/v % EC + | 1M LiFSI | 4.1 |
| | | 2 wt % FEC | | |
| 37 | 10 | 70 v/v % EC/DEC (3:7 vol) + | 1M LiFSI | 4.2 |
| | | 2 wt % FEC | | |
| 38 | 10 | 50 v/v % EC/DEC (3:7 vol) + | 1M LiFSI | 4.3 |
| | | 2 wt % FEC | | |
| 39 | 10 | 30 v/v % EC/DEC (3:7 vol) + | 1M LiFSI | 4.7 |
| | | 2 wt % FEC | | |
| 40 | 10 | 20 v/v % EC/DEC (3:7 vol) + | 1M LiFSI | 5.2 |
| | | 2 wt % FEC | | |
| 41 | 14 | 75 v/v % EC/DEC (3:7 vol) + | 1M LiFSI | 4.2 |
| | | 2 wt % FEC | | |
| 42 | 14 | 50 v/v % EC/DEC (3:7 vol) + | 1M LiFSI | 4.4 |
| | | 2 wt % FEC | | |
| 43 | 14 | 25 v/v % EC/DEC (3:7 vol) + | 1M LiFSI | 4.4 |
| | | 2 wt % FEC | | |
| 44 | 15 | 70 v/v % EC/DEC (3:7 vol) + | 1M LiFSI | 4.4 |
| | | 2 wt % FEC | | |
| 45 | 15 | 50 v/v % EC/DEC (3:7 vol) + | 1M LiFSI | 4.4 |
| | | 2 wt % FEC | | |
| 46 | 15 | 30 v/v % EC/DEC (3:7 vol) + | 1M LiFSI | 4.4 |
| | | 2 wt % FEC | | |
| 47 | 15 | 20 v/v % EC/DEC (3:7 vol) + | 1M LiFSI | >5.5 |
| | | 2 wt % FEC | | |
| 48 | 23 | 2 wt % FEC | 1M LiFSI | 5.1 |
| 49 | 24 | 2 wt % FEC | 1M LiFSI | 5.1 |
| 50 | 25 | 2 wt % FEC | 1M LiFSI | 5.4 |
| 51 | 26 | 2 wt % FEC | 1M LiFSI | 4.8 |
| 52 | 27 | 2 wt % FEC | 1M LiFSI | 4.9 |
| 53 | 28 | 2 wt % FEC | 1M LiFSI | 5.2 |
| 54 | 29 | 2 wt % FEC | 1M LiFSI | 4.9 |

### Button Cell Charge-Discharge Tests

### Example 55 (Comparative): Unsuccessful Charging and Discharging of LCO in LiFSI-EC-DEC Electrolyte

An LCO cathode material was prepared using a mixture of LCO, VGCF (vapour grown carbon nanotubes), carbon black and polyvinylidene fluoride (PVDF) in a ratio 89:3:3:5 by weight in N-methyl-2-pyrrolidone (NMP). The mixture was then coated on a 15 μm thick non-coated aluminum current collector, provided by UACJ. The electrode material was calendered, cut into discs and dried at 120° C. in a vacuum oven for 12 h before use.

A button cell was assembled using one of the above-described discs (16 mm diameter) of LCO as a cathode, Celgard Q20S1HX as separator membrane, the electrolyte of comparative example 5, and a 16 mm, 200 μm thick disc of lithium metal, provided by China Energy Lithium Co., LTD., as an anode.

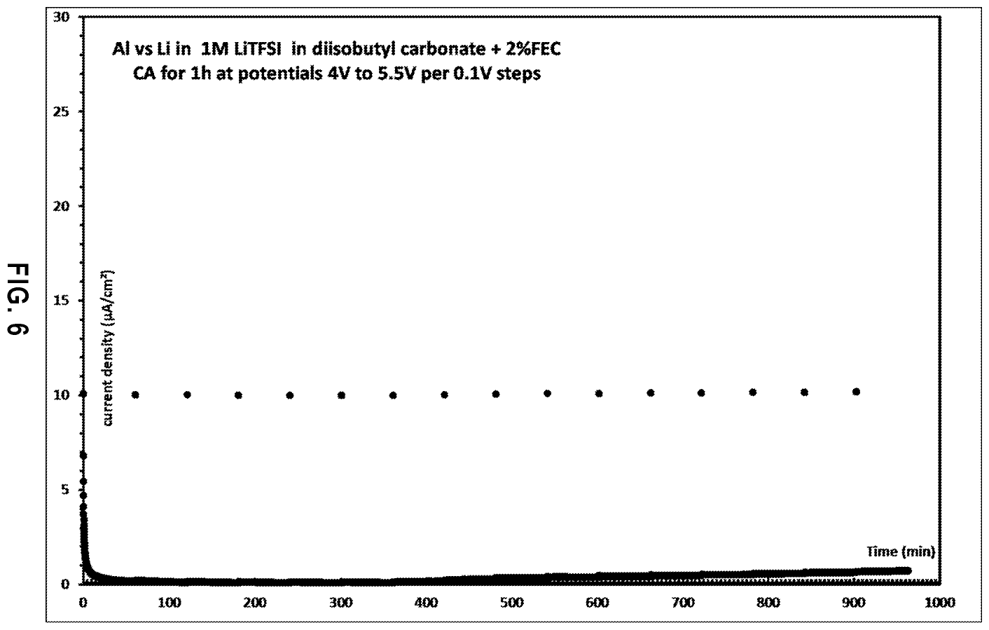

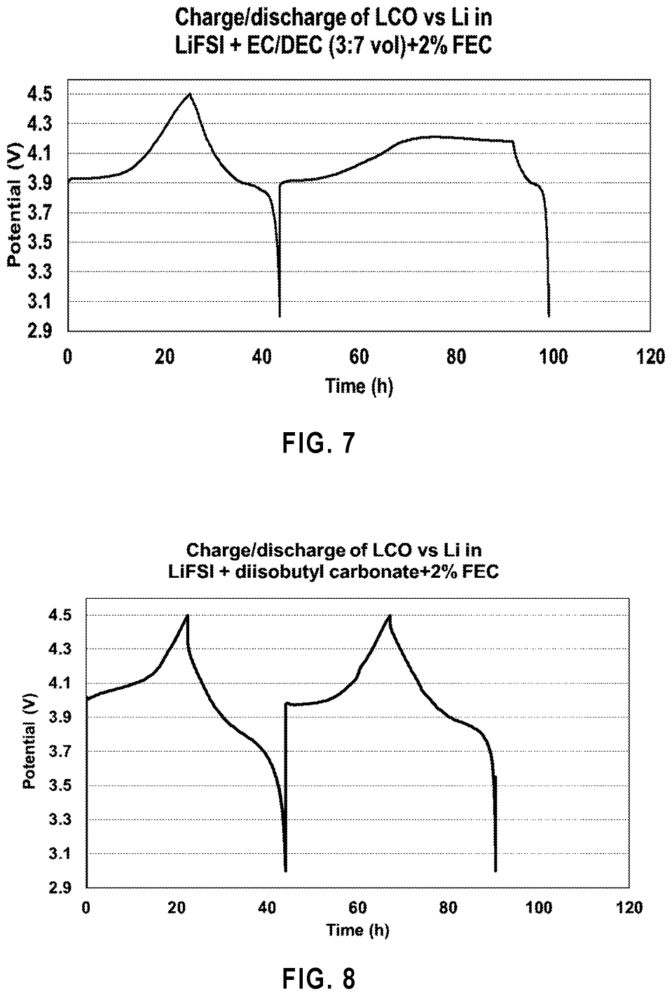

The cell was used for probing the charging and discharging between 3 and 4.5 V at C/24 rate. The results of this experiment can be seen in FIG. 7. The first charge/discharge cycle has a normal shape, but during second charging an unexpected plateau appears at 4.2 V. This plateau could be attributed to the anodic dissolution of aluminum current collector, which leads to a loss of charge and a very low discharge capacity. Accordingly, this electrolyte does not support the operation of an LCO electrode.

### Example 56: Successful Charging and Discharging of LCO in LiFSI-Diisobutyl Carbonate Electrolyte

A button cell was assembled using a disc of 16 mm diameter LCO as a cathode (prepared using the process described in Example 55), Celgard Q20S1HX as separator membrane, the electrolyte of example 8, and a 16 mm, 200 μm thick disc of lithium metal, provided by China Energy Lithium Co., LTD., as an anode.

The cell was used for probing the charging and discharging between 3 and 4.5 V at C/24 rate. The results of this experiment can be seen in FIG. 8. The charge-discharge curves are deformed, but one cannot detect any sign of a parasitic process, which would manifest as a plateau similar to the second cycle in FIG. 7. Accordingly, this electrolyte can support the operation of an LCO electrode, potentially with some additives to further improve its performance (which is shown in Example 57).

### Example 57: Successful Charging and Discharging of LCO in LiFSI-EC-Diisobutyl Carbonate Electrolyte

The electrolyte of example 33 (i.e. a 1M solution of LiFSI (Nippon Shokubai) in a 1:9 mixture by volume of ethylene carbonate (EC) and diisobutyl carbonate (solvent no. 10), respectively, to which 2% of fluoroethylene carbonate was added) was prepared. Note that this electrolyte is similar to the electrolyte of example 56 except that solvent no. 10 was replaced by a 1:9 mixture by volume of EC and solvent no. 10. In other words, EC is used as an additive herein.

A button cell was assembled using a disc of 16 mm diameter LCO coated on a 15 μm thick aluminum current collector (prepared using the process described in Example 55), provided by UACJ, as a cathode; Celgard Q20S1HX as a separator membrane; the preceding LiFSI-EC-diisobutyl carbonate electrolyte; and a 16 mm, 200 μm thick disc of lithium metal, provided by China Energy Lithium Co., LTD., as an anode.

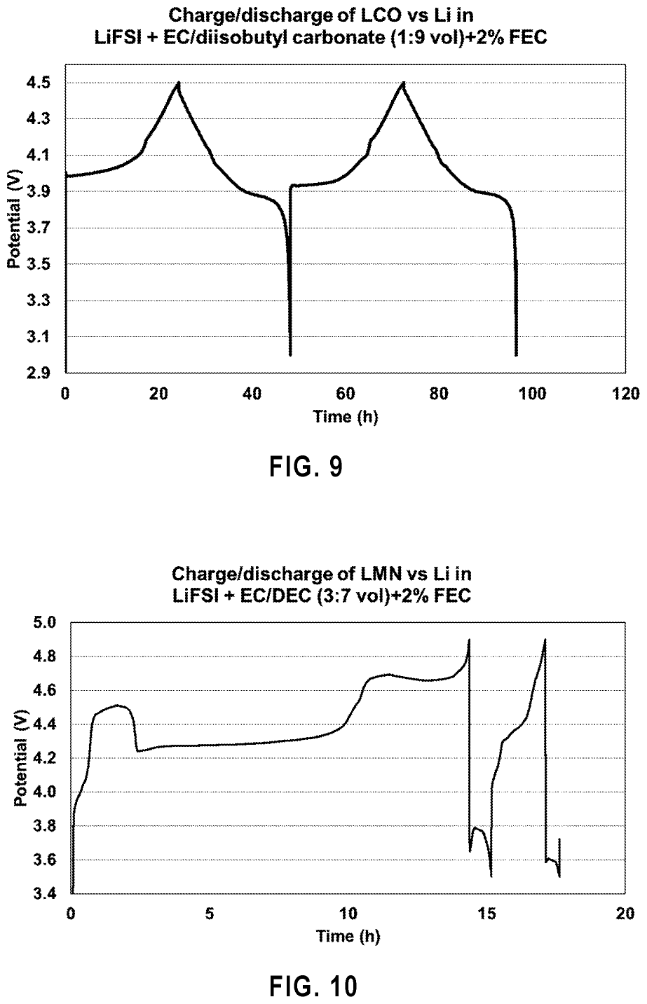

The cell was used for probing the charging and discharging between 3 and 4.5 V at C/24 rate. The results of this experiment can be seen in FIG. 9. The charge-discharge curves have a normal shape and one cannot detect any sign of the parasitic process which would manifest as a plateau similar to the second cycle in FIG. 7. Accordingly, this electrolyte can support quite well the operation of an LCO electrode.

### Example 58 (Comparative): Unsuccessful Charging and Discharging of LMN in LiFSI-EC-DEC Electrolyte

A LiMn_{3/2}Ni_{1/2}O₄ (LMN) cathode material was prepared using a mixture of LMN, VGCF (vapour grown carbon nanotubes), carbon black and polyvinylidene fluoride (PVDF) in a ratio of 94:1.5:1.5:3 by weight in NMP. The mixture was then coated on a 15 μm thickness of non-coated aluminum current collector, provided by UACJ. The electrode material was calendered, cut into discs and dried at 120° C. in a vacuum oven for 12 h before use.

A button cell was assembled using a disc of 16 mm diameter LMN as a cathode, Celgard Q20S1HX as a separator membrane, the electrolyte of comparative example 5, and a 16 mm, 200 μm thick disc of lithium metal, provided by China Energy Lithium Co., LTD., as an anode.

The cell was used for probing the charging and discharging between 3.5 and 4.9 V at C/24 rate. The results of this experiment can be seen in FIG. 10. The first charge cycle shows an abnormal shape. First, the potential increases to approximately 4.5 V but then decreases down to an unexpected plateau at approximately 4.3 V. This plateau could be attributed to the anodic dissolution of the aluminum current collector, which lead to the extreme malfunctioning of the battery, as not even one normal cycle could be performed. Therefore, this electrolyte cannot be used at all with an LMN electrode.

### Example 59: Successful Charging and Discharging of LMN in LiFSI-Diisobutyl Carbonate Electrolyte

A button cell was assembled using a disc of 16 mm diameter LMN as a cathode (prepared using the process described in Example 58), Celgard Q20S1HX as separator membrane, the electrolyte of example 8, and a 16 mm, 200 μm thick disc of lithium metal, provided by China Energy Lithium Co., LTD., as an anode.

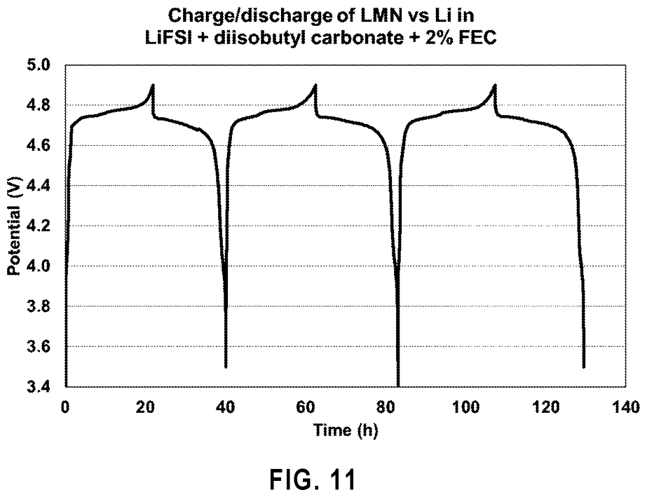

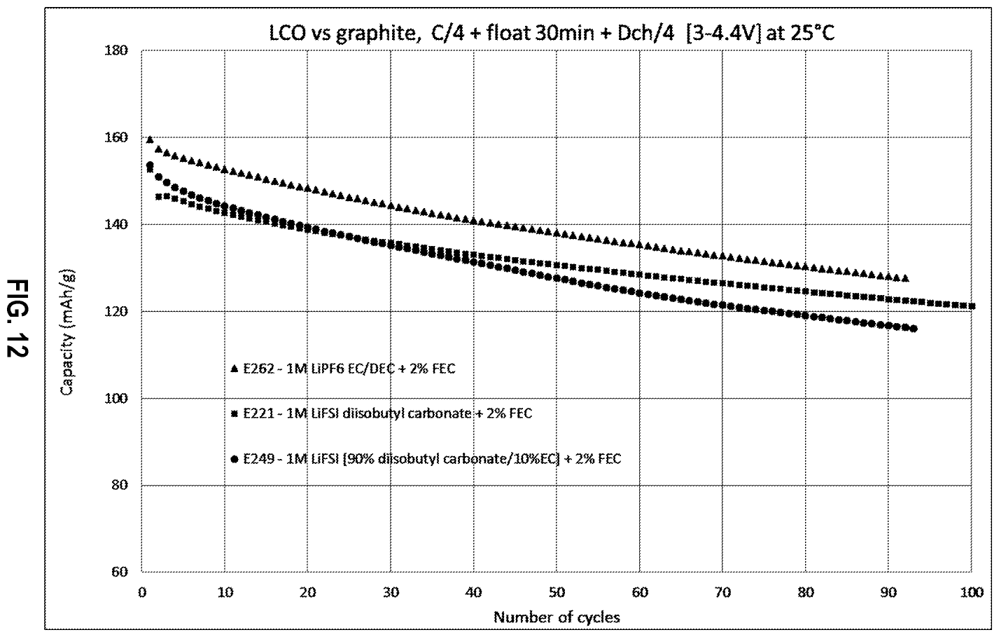

The cell was used for probing the charging and discharging between 3.5 and 4.9V at C/24 rate. The results of this experiment can be seen in FIG. 11. The charge-discharge curves appear normal and one cannot detect any sign of parasitic process, which would manifest as a plateau similar to that found in FIG. 10. This electrolyte can therefore support the operation of an LMN electrode, possibly with some additives to further improve its performance.

### Example 60: Extended Temperature Range of a Diisobutyl Carbonate-Based Electrolyte Compared to Conventional Solvent

A digital scanning calorimetry experiment was performed on the electrolyte of example 33 and on the electrolyte of comparative example 5.

The electrolyte of comparative example 5 exhibited a melting pant of −10° C. and a glass transition point of −111° C. In contrast, the electrolyte of the invention showed no melting point and a glass transition point of 98° C. In other words, the electrolyte of example 33 stayed in liquid form and eventually in amorphous solid form, without crystallizing, until it reached its glass transition point of −98° C. This indicates that the electrolyte of the invention can be used at lower temperatures than conventional electrolytes without crystallisation.

### Example 61: Full Li-Ion Cell

Electrolytes from example 8 (LIFSI in diisobutyl carbonate, 2% of FEC) and example 33 (LiFSI in 90% diisobutyl carbonate:10% EC, 2% of FEC) and a conventional electrolyte of 1 M LiPF₆ in EC/DEC (3:7 vol) with 2% of FEC were tested.

A graphite electrode was prepared by Cumstomcells Company by mixing 96% of modified graphite (SMG), 2% of water-based binder, and 2% of electronic conductivity enhancer in water; coating the mixture onto a 14 μm thick copper foil; drying it and calendering it. The resulting electrode material was cut into discs and dried at 120° C. in a vacuum oven for 12 h before use.

Li-ion button cells were assembled using a disc of 16 mm diameter LCO coated on 15 μm thick aluminum current collector (as in example 55), provided by UACJ, as a cathode; Celgard Q20S1HX as separator membrane; one of the above-listed electrolytes; and the above-prepared 16 mm disc of graphite electrode as an anode.

The cells were subjected to three formation cycles—the charging and discharging between 3 and 4.4 V at C/24 rate. After that, the cells were subjected to long term cycling with charging at C/4, followed by a 30 min float at 4.4 V and C/4 discharge. The results of this experiment—the discharge capacity of the cells versus cycle number—can be seen in FIG. 12. The LiPF₆ electrolyte provides the highest starting discharge capacity, but then one can observe relatively linear diminution of the capacity over cycle number. LiFSI in pure diisobutyl carbonate has approximately 10% less of the starting capacity, but degradation of the capacity is slower than in the case of LiPF₆. The addition of 10% of EC to pure diisobutyl carbonate electrolyte increases the starting capacity, but the speed of degradation approaches to that of LiPF₆.

With this experiment, the utilisation electrolytes prepared of LIFSI in the solvents of the present invention in high voltage Li-ion batteries has been demonstrated, while the utilisation of LiFSI in conventional solvents is not possible for this battery system.

The scope of the claims should not be limited by the preferred embodiments set forth in the examples, but should be given the broadest interpretation consistent with the description as a whole.

### REFERENCES

The present description refers to a number of documents, the content of which is herein incorporated by reference in their entirety. These documents include, but are not limited to, the following:
U.S. Pat. No. 3,359,296
U.S. Pat. No. 5,072,040
U.S. Pat. No. 5,292,601
U.S. Pat. No. 5,446,134
U.S. Pat. No. 9,698,447
US 2002/122988
US 2005/241145
EP 858994
WO 2017/221908
WO 2018072024
Beran, M.; Příhoda, J.; Žák, Z.; Černík, M. Polyhedron 2006, 25, 1292.
Beran, M.; Příhoda, J.; Taraba, J. Polyhedron 2010, 29, 991.
Han, H.-B.; Zhou, S.-S.; Zhang, D.-J.; Feng, S.-W.; Li, L.-F.; Liu, K.; Feng, W.-F.; Nie, J.; Li, H.; Huang, X.-J.; Armand, M.; Zhou, Z.-B. J. Power Sources 2011, 196, 3623.
Meister, P.; Qi, X.; Kloepsch, R.; Krämer, E.; Streipert, B.; Winter, M.; Placke, T. ChemSusChem 2017, 10, 804.
Kraemer, E.; Passerini, S.; Winter, M. ECS Electrochem. Lett 2012, 1, C9.
Park, K.; Yu, S.; Lee, C.; Lee, H. J. Power Sources 2015, 296, 197.
Xia, L.; Jiang, Y.; Pan, Y.; Li, S.; Wang, J.; He, Y.; Xia, Y.; Liu, Z.; Chen, G. Z. ChemistrySelect 2018, 3, 1954.
Yamada, Y.; Chiang, C. H.; Sodeyama, K.; Wang, J.; Tateyama, Y.; Yamada, A. ChemElectroChem 2015, 2, 1687.
Flamme, B.; Rodriguez Garcia, G.; Weil, M.; Haddad, M.; Phansavath, P.; Ratovelomanana-Vidal, V.; Chagnes, A. Green Chemistry 2017, 19, 1828.
Shaikh, A-A G.; Sivaram, S. Chem. Rev. 1996, 96, 951, Parrish, J. P.; Salvatore, R. N.; Jung, K. W. Tetrahedron 2000, 56, 8207.
Buysch, H.-J. In Ullmann's Encyclopedia of Industrial Chemistry; Wiley-VCH Verlag GmbH & Co. KGaA: 2000; Vol. 7, p 45, doi: 10.1002114356007.a05_197
Tundo, P.; Aricò, F.; Rosamilia Anthony, E.; Rigo, M.; Maranzana, A; Tonachini, G. In Pure Appl. Chem. 2009; Vol. 81, p 1971.
Kenar, J. A; Knothe, G.; Copes, A. L. J. Am. Oil Chem. Soc. 2004, 81, 285.
Chen, Z.; Zhang, Z.; Amine, K. In Advanced Fluoride-Based Materials for Energy Conversion; Groult, H., Ed.; Elsevier: 2015, p 1.

1. A metal or metal-ion battery comprising:
(a) a cathode comprising an aluminum current collector and having an upper potential limit of about 4.2 V or more vs a Li-metal reference electrode,
(b) an anode,
(c) a separator membrane separating the anode and the cathode, and
(d) a low-corrosiveness non-aqueous electrolyte in contact with the anode and the cathode,
wherein the battery has an upper voltage limit of about 4.2 V or more,
wherein anodic dissolution of aluminum in the aluminum current collector is suppressed during battery operation at voltages up to said upper voltage limit, and
wherein the electrolyte comprises, as a solvent, a carbonate compound of formula (I): 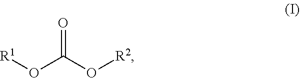
wherein:
R¹ represents a C₃-C₂₄ alkyl, a C₃-C₂₄ alkoxyalkyl, a C₃-C₂₄ ω-O-alkyl oligo(ethylene glycol),
or a C₄-C₂₄ ω-O-alkyl oligo(propylene glycol), and
R² represents a C₁-C₂₄ alkyl, a C₁-C₂₄ haloalkyl, a C₂-C₂₄ alkoxyalkyl, a C₂-C₂₄ alkyloyloxyalkyl, a C₃-C₂₄ alkoxycarbonylalkyl, a C₁-C₂₄ cyanoalkyl, a C₁-C₂₄ thiocyanatoalkyl, a C₃-C₂₄ trialkylsilyl, a C₄-C₂₄ trialkylsilylalkyl, a C₄-C₂₄ trialkylsilyloxyalkyl, a C₃-C₂₄ ω-O-alkyl oligo(ethylene glycol), a C₄-C₂₄ ω-O-alkyl oligo(propylene glycol), a C₅-C₂₄ ω-O-trialkylsilyl oligo(ethylene glycol), or a C₆-C₂₄ ω-O-trialkylsilyl oligo(propylene glycol),
and a conducting salt dissolved in said solvent.

2. The battery of claim 1, wherein the upper potential limit of the cathode is about 4.4 V or more, preferably about 4.6 V or more, about 4.8 V or more, about 5.0 V or more, about 5.2 V or more, about 5.4 V or more, or about 5.5 V or more, vs a Li-metal reference electrode.

3. The battery of claim 1, wherein the upper voltage limit of the battery is about 4.4 V or more, preferably about 4.6 V or more, more preferably about 4.8 V or more, yet more preferably about 5.0 V or more, even more preferably about 5.2 V or more, more preferably about 5.4 V or more, or most preferably about 5.5 V or more.

4. The battery of claim 1, wherein R¹ represents a C₃-C₂₄ alkyl or a C₃-C₂₄ ω-O-alkyl oligo(ethylene glycol), preferably a C₃-C₂₄ alkyl.

5. The battery of claim 1, wherein R² represents a C₁-C₂₄ alkyl, a C₂-C₂₄ alkoxyalkyl, a C₁-C₂₄ cyanoalkyl, a C₄-C₂₄ trialkylsilyloxyalkyl, a C₅-C₂₄ ω-O-trialkylsilyl oligo(ethylene glycol), or a C₃-C₂₄ ω-O-alkyl oligo(ethylene glycol), preferably a C₁-C₂₄ alkyl.

6. The battery of claim 1, wherein the sum of the carbon atoms in R¹ and R² is:
5 or more, preferably 6 or more, more preferably 7 or more, yet more preferably 8 or more, and most preferably 9 or more, and/or
24 or less, preferably 20 or less, more preferably 16 or less, yet more preferably 14 or less, even more preferably 12 or less, and most preferably 10 or less.

7-17. (canceled)

18. The battery of claim 1, wherein the carbonate compound of formula (I) is didodecyl carbonate, dibutyl carbonate, dipropyl carbonate, methyl propyl carbonate, diisopropyl carbonate, isopropyl methyl carbonate, ethyl dodecyl carbonate, ethyl propyl carbonate, ethyl isopropyl carbonate, diisobutyl carbonate, isobutyl methyl carbonate, dipentyl carbonate, methyl pentyl carbonate, di(2-ethylhexyl) carbonate, 2-ethylhexyl methyl carbonate, methyl 2-pentyl carbonate, di(2-pentyl) carbonate, 2-butyl methyl carbonate, di(2-butyl) carbonate, 2-ethylbutyl methyl carbonate, di(2-ethylbutyl) carbonate, isobutyl isopropyl carbonate, 2-cyanoethyl butyl carbonate, 2-methoxyethyl isobutyl carbonate, (2-trimethylsilyloxy)ethyl butyl carbonate, di(2-methoxyethyl) carbonate, 2-isopropoxyethyl methyl carbonate, di(2-isopropoxyethyl) carbonate, or di(2-(2-methoxyethoxy)ethyl) carbonate.

19. (canceled)

20. The battery of claim 1, wherein the compound of formula (I) is didodecyl carbonate, di(2-ethylhexyl) carbonate, 2-ethylhexyl methyl carbonate, ethyl dodecyl carbonate, or diisobutyl carbonate, preferably diisobutyl carbonate.

21. The battery of claim 1, wherein the conducting salt is:
LiClO₄;
LiP(CN)_{α}F_{6-α}, where α is an integer from 0 to 6, preferably LiPF₆;
LiB(CN)_{β}F_{4-β}, where β is an integer from 0 to 4, preferably LiBF₄;
LiP(CₙF₂ₙ₊₁)_{γ}F_{6-γ}, where n is an integer from 1 to 20, and γ is an integer from 1 to 6;
LiB(CₙF₂ₙ₊₁)_{δ}F_{4-δ}, where n is an integer from 1 to 20, and δ is an integer from 1 to 4;
Li₂Si(CₙF₂ₙ₊₁)_{ε}F_{6-ε}, where n is an integer from 1 to 20, and ε is an integer from 0 to 6;
lithium bisoxalato borate;
lithium difluorooxalatoborate; or
a compound represented by one of the following general formulas: 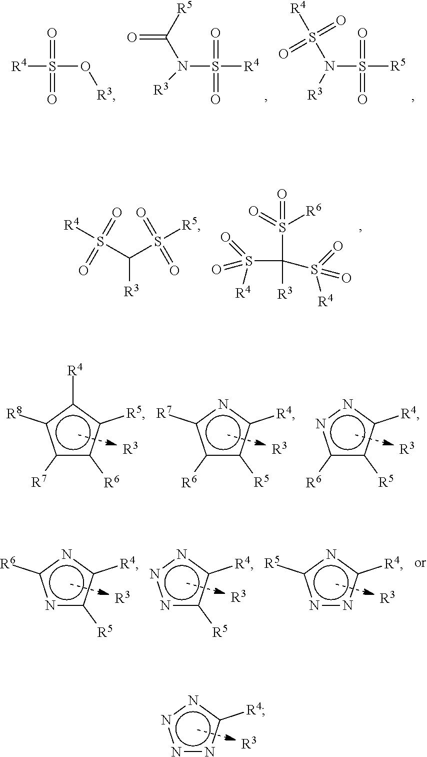
wherein:
R³ represents: Li, Na, K, Rb, Cs, Be, Mg, Ca, Sr, Ba, Al, hydrogen, or an organic cation; and
R⁴, R⁵, R⁶, R⁷, R⁸ represent: cyano, fluorine, chlorine, branched or linear alkyl radical with 1-24 carbon atoms, perfluorinated linear alkyl radical with 1-24 carbon atoms, aryl, heteroaryl, perfluorinated aryl, or heteroaryl;
or a derivative thereof.

22. The battery of claim 1, wherein the conducting salt is a sulfonylamide salt.

23-32. (canceled)

33. The battery of claim 1, wherein the carbonate compound of formula (I) is the only solvent in the electrolyte.

34-36. (canceled)

37. The battery of claim 1, wherein the electrolyte is free of corrosion inhibitors.

38-43. (canceled)

44. The battery of claim 1, being a sodium battery, a sodium-ion battery, a potassium battery, a potassium-ion battery, a magnesium battery, a magnesium-ion battery, an aluminum battery, or an aluminum ion battery.

45. A carbonate compound of formula (I): 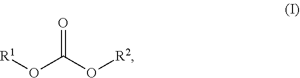
wherein
R¹ represents a C₃-C₂₄ alkyl, a C₃-C₂₄ alkoxyalkyl, a C₃-C₂₄ ω-O-alkyl oligo(ethylene glycol), or a C₄-C₂₄ ω-O-alkyl oligo(propylene glycol), and
R² represents a C₁-C₂₄ alkyl, a C₁-C₂₄ haloalkyl, a C₂-C₂₄ alkoxyalkyl, a C₂-C₂₄ alkyloyloxyalkyl, a C₃-C₂₄ alkoxycarbonylalkyl, a C₁-C₂₄ cyanoalkyl, a C₁-C₂₄ thiocyanatoalkyl, a C₃-C₂₄ trialkylsilyl, a C₄-C₂₄ trialkylsilylalkyl, a C₄-C₂₄ trialkylsilyloxyalkyl, a C₃-C₂₄ ω-O-alkyl oligo(ethylene glycol), a C₄-C₂₄ ω-O-alkyl oligo(propylene glycol), a C₅-C₂₄ ω-O-silyl oligo(ethylene glycol), or a C₆-C₂₄ ω-O-silyl oligo(propylene glycol),
with proviso that when R² is a C₁-C₉ alkyl, R¹ represents a C₁₀-C₂₄ alkyl, a C₃-C₂₄ alkoxyalkyl, a C₃-C₂₄ ω-O-alkyl oligo(ethylene glycol), or a C₄-C₂₄ ω-O-alkyl oligo(propylene glycol).

46. The carbonate compound of claim 45, wherein, when R² is a C₁-C₉ alkyl, R¹ represents a C₃-C₂₄ alkoxyalkyl, a C₃-C₂₄ ω-O-alkyl oligo(ethylene glycol), or a C₄-C₂₄ ω-O-alkyl oligo(propylene glycol).

47. The carbonate compound of claim 45, wherein the sum of the carbon atoms in R¹ and R² is:
5 or more, preferably 6 or more, more preferably 7 or more, yet more preferably 8 or more, and most preferably 9 or more, and/or
24 or less, preferably 20 or less, more preferably 16 or less, yet more preferably 14 or less, even more preferably 12 or less, and most preferably 10 or less.

48-49. (canceled)

50. The carbonate compound of claim 45, wherein:
R¹ represents a C₁₀-C₂₄ alkyl (preferably C₁₂-C₂₄ alkyl, more preferably C₁₄-C₂₄ alkyl) and R² represents a C₁-C₂₄ alkyl, or
R¹ represents a C₃-C₂₄ alkyl and R² represents a C₁-C₂₄ cyanoalkyl, or
R¹ represents a C₃-C₂₄ alkyl and R² represents a C₂-C₂₄ alkoxyalkyl, or
R¹ represents a C₃-C₂₄ alkoxyalkyl and R² represents a C₁-C₂₄ alkyl, or
R¹ and R² both represent a C₃-C₂₄ alkoxyalkyl, or
R¹ represents a C₃-C₂₄ alkyl and R² represents a C₄-C₂₄ trialkylsilyloxyalkyl, or
R¹ and R² both represent a C₃-C₂₄ ω-O-alkyl oligo(ethylene glycol).

51-60. (canceled)

61. The carbonate compound of claim 45, wherein the carbonate compound of formula (I) is didodecyl carbonate, ethyl dodecyl carbonate, 2-cyanoethyl butyl carbonate, 2-methoxyethyl isobutyl carbonate, (2-trimethylsilyloxy)ethyl butyl carbonate, di(2-methoxyethyl) carbonate, 2-isopropoxyethyl methyl carbonate, di(2-isopropoxyethyl) carbonate, or di(2-(2-methoxyethoxy)ethyl) carbonate.

62-63. (canceled)
